(19) United States
(12) Patent Application Publication
(10) Pub. No.: US 2023/0398204 A1
(43) Pub. Date: Dec. 14, 2023

(54) Coronavirus Spike Glycoprotein With Improved Expression and Stability

(71) Applicant: La Jolla Institute for Immunology, La Jolla, CA (US)

(72) Inventors: Erica Ollman Saphire, La Jolla, CA (US); Eduardo Olmedillas, La Jolla, CA (US); Colin Mann, La Jolla, CA (US)

(21) Appl. No.: 18/031,800

(22) PCT Filed: Oct. 21, 2020
(86) PCT No.: PCT/US2021/056037

§ 371 Date: Apr. 13, 2023

Related U.S. Application Data
(60) Provisional application No. 63/094,451, filed on Oct. 21, 2020.
(60) Provisional application No. 63/170,236, filed on Apr. 2, 2021.
(60) Provisional application No. 63/212,814, filed on Jun. 21, 2021.
(60) Provisional application No. 63/234,497, filed on Aug. 18, 2021.

(51) Int. Cl. A61K 39/215; C07K 14/005

(52) U.S. Cl. CPC A61K 39/215; A61K 2039/53; C07K 14/005

(57) ABSTRACT

The present invention includes a mutant coronavirus spike protein, methods of making and using, vaccines, vectors and nucleic acids, comprising at least one of the following modifications: a short flexible peptide linker or a rigid peptide linker in place of the furin cleavage site loop to genetically link an 51 and S2 subunit; at least one additional disulfide bond; or 1, 2, 3, 4, or 5 proline mutations for greater trimeric stability, wherein the resulting mutant coronavirus spike protein has at least one of: a higher stability or a higher level of expression when compared to a non-modified coronavirus spike protein. In one example, the coronavirus is SARS, MERS, 229E (alpha), NL63 (alpha), OC43 (beta), HKU1 (beta), SARS-CoV-2, or an emerging variant thereof. Current SARS-CoV-2 variants include, e.g., B.1.1.7, B.1.1.7 with E484K, B.1.135, B.1.351, P.1, B.1.427, D614G, B.1.1351, or B.1.429, Lambda (i.e., C.37), Mu (i.e. B.1.621), and others.

### CROSS-REFERENCE TO RELATED APPLICATIONS

This application claims priority to U.S. Provisional Patent Application Nos. 63/094,451, filed Oct. 21, 2020, 63/170,236 filed Apr. 2, 2021, 63/212,814 filed Jun. 21, 2021 and 63/234,497, filed Aug. 18, 2021, the entire contents of each of which are incorporated herein by reference.

### TECHNICAL FIELD OF THE INVENTION

The present invention relates in general to the field of a coronavirus spike glycoprotein with improved expression and stability, and more particularly, to a structure-based design and characterization of a SARS-CoV-2 spike glycoprotein with improved expression and stability.

### STATEMENT OF FEDERALLY FUNDED RESEARCH

Not applicable.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on ______, 2021, is named ______.txt and is ______ bytes in size.

### BACKGROUND OF THE INVENTION

Without limiting the scope of the invention, its background is described in connection with SARS-CoV-2.

The worldwide spread of SARS-CoV-2 in the human population resulted in the ongoing COVID-19 pandemic that has already caused more than 241 million infections and more than 4.9 million deaths. To initiate infection, the SARS-CoV-2 spike (S) glycoprotein promotes binding to ACE2 located on the surface of the host cell, initiating a cascade of conformational changes in the protein that drives from a metastable pre-fusion conformation to a stable post-fusion conformation. That reorganization of the protein exposes the fusion peptide and final conduct to a fusion between the viral and host membranes driven by the S2 chain of the proprotein. Given its external location on the virus membrane and its functionality, SARS-CoV-2 spike ‘5’ protein in its pre-fusion state is the main target of neutralizing antibodies and therefore the main target of the design of safe and effective vaccines. Moreover, since this epidemic is global, a vaccine is urgently needed that can be transported and used everywhere, including low-to-middle-income countries (LMIC). Stability and conformational dynamics of the spike-based vaccine are fundamental factors for the development of vaccines, diagnostics, and countermeasures against this virus. What is needed are improved antigenic proteins that are more stable for storage, manufacturing, freeze/thaw, and lyophilization/resuspension.

### SUMMARY OF THE INVENTION

As embodied and broadly described herein, an aspect of the present disclosure relates to a mutant coronavirus spike protein comprising at least one of the following modifications: (1) a short flexible peptide linker or a rigid peptide linker in place of a furin cleavage site loop to genetically link an 51 and S2 subunit; (2) at least one additional disulfide bond; or (3) 1, 2, 3, 4, or 5 proline mutations for greater trimeric stability, wherein the resulting mutant coronavirus spike protein has at least one of: a higher stability or a higher level of expression when compared to a non-modified coronavirus spike protein, and a glycan shield similar to the virion. In one aspect, the furin cleavage site loop is at position 676-690. In another aspect, the linker is selected from at least one of: GGS (SEQ ID NO:34), GP (SEQ ID NO:35), GPGP (SEQ ID NO:36), GGSGGS (SEQ ID NO:37), or GGGSGGGS (SEQ ID NO:38). In another aspect, the coronavirus is a SARS-CoV-2 coronavirus with 1, 2, 3, 4, or 5 proline mutations are selected from F817P, A892P, A899P, A942P, P986K, K986P, V987P, and P987V, and, e.g., F817P, A892P, A899P, A942P, K986P, and V987P. In another aspect, the coronavirus is a SARS-CoV-2 coronavirus with at least one additional disulfide bond is selected from F43 C-G566C, G413 C-P987C, Y707C-T883C, G1035C-V1040C, A701C-Q787C, G667C-L864C, V382C-R983C, or I712C-I816C. In another aspect, the coronavirus is a SARS-CoV-2 coronavirus with wherein proline mutations are not K986P and V987P mutations. In another aspect, the coronavirus is a SARS-CoV-2 coronavirus with at least one addition disulfide bond links the S2 to S2′ subunit, the 51 to S2 subunit, or the 51 to S2′ subunit. In another aspect, the higher stability is selected from: increased temperature stability (including the ability to store the composition at room temperature), increased freeze/thaw stability, or increased lyophilization/resuspension stability. In another aspect, the mutant coronavirus spike protein further comprising a purification peptide at an amino-terminus, a carboxy-terminus, or both. In another aspect, the mutant coronavirus is a SARS-CoV-2 spike protein is selected from SEQ ID NOS:1 to 33. In another aspect, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages). In another aspect, the mutant coronavirus spike proteins are formed into dimers, trimers, multimers, or nanoparticles. In another aspect, the nanoparticles comprise ferritin nanoparticles, polymeric nanoparticles, or both.

As embodied and broadly described herein, an alternative aspect of the present disclosure relates to a method of making a mutant coronavirus spike protein comprising: obtaining a nucleic acid sequence the encodes a coronavirus spike protein; and modifying the nucleic acid sequence of the coronavirus spike protein to mutate an amino acid sequence thereof by at least one of: linking the S1/S2 subunits of a coronavirus spike protein, by deleting a furin cleavage site loop and adding a short flexible peptide linker or a rigid peptide linker; adding at least one additional disulfide bond; or adding 1, 2, 3, 4, or 5 proline mutations for greater trimeric stability, wherein the resulting mutant coronavirus spike protein has at least one of: higher stability or level of expression than a non-modified coronavirus spike protein, and a glycan shield similar to the virion. In one aspect, the method further comprises the step of expressing the mutant coronavirus spike protein in a bacteria, fungi, mammalian cell, avian cell, insect cell, or plant cell. In another aspect, the furin cleavage site loop is at position 676-690. In another aspect, the linker is selected from at least one of: GGS (SEQ ID NO:34), GP (SEQ ID NO:35), GPGP (SEQ ID NO:36), GGSGGS (SEQ ID NO:37), or GGGSGGGS (SEQ ID NO:38). In another aspect, the coronavirus is a SARS-CoV-2 coronavirus with 1, 2, 3, 4, or 5 proline mutations are selected from F817P, A892P, A899P, A942P, P986K, K986P, V987P, and P987V, and, e.g., F817P, A892P, A899P, A942P, K986P, and V987P. In additional aspects, the 1, 2, 3, 4, or 5 proline mutations are selected from F817P, A892P, A899P, A942P, K986P and V987P. In another aspect, the coronavirus is a SARS-CoV-2 coronavirus with at least one additional disulfide bond is selected from F43C-G566C, G413C-P987C, Y707C-T883C, G1035C-V1040C, A701C-Q787C, G667C-L864C, V382C-R983C, or I712C-I816C. In another aspect, the coronavirus is a SARS-CoV-2 coronavirus wherein proline mutations are not K986P and V987P mutations. In another aspect, the coronavirus is a SARS-CoV-2 coronavirus with at least one addition disulfide bond links the S2 to S2′ subunit, the Si to S2 subunit, or the Si to S2′ subunit. In another aspect, the higher stability is selected from: increased temperature stability (including the ability to store the composition at room temperature), increased freeze/thaw stability, or increased lyophilization/resuspension stability. In another aspect, the method further comprises a purification peptide at an amino-terminus, a carboxy-terminus, or both. In another aspect, the mutant coronavirus is a SARS-CoV-2 spike protein is selected from SEQ ID NOS:1 to 33. In another aspect, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages). In another aspect, the mutant coronavirus spike proteins are formed into dimers, trimers, multimers, or nanoparticles. In another aspect, the nanoparticles comprise ferritin nanoparticles, polymeric nanoparticles, or both.

As embodied and broadly described herein, an alternative aspect of the present disclosure relates to a vaccine comprising: a mutant coronavirus spike protein comprising at least one of the following modifications: a short flexible peptide linker or a rigid peptide linker in place of the furin cleavage site loop to genetically link an Si and S2 subunit; at least one addition disulfide bond; or 1, 2, 3, 4, or 5 proline mutations for greater trimeric stability, wherein the resulting mutant coronavirus spike protein has at least one of: a higher stability or a higher level of expression when compared to a non-modified coronavirus spike protein, and a glycan shield similar to the virion; and one or more pharmaceutically acceptable excipients or carriers. In one aspect, the vaccine further comprises one or more adjuvants. In another aspect, the mutant coronavirus is a SARS-CoV-2 spike protein is selected from SEQ ID NOS:1 to 33. In another aspect, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages). In another aspect, the mutant coronavirus spike proteins are formed into dimers, trimers, multimers, or nanoparticles. In another aspect, the nanoparticles comprise ferritin nanoparticles, polymeric nanoparticles, or both.

As embodied and broadly described herein, an alternative aspect of the present disclosure relates to a method of immunizing a subject in need thereof, the method comprising: identifying a subject in need of an immunization; and exposing the subject to a mutant coronavirus spike protein comprising at least one of the following modifications: a short flexible peptide linker or a rigid peptide linker in place of the furin cleavage site loop to genetically link an S1 and S2 subunit; at least one additional disulfide bond; or 1, 2, 3, 4, or 5 proline mutations for greater trimeric stability, wherein the resulting mutant coronavirus spike protein has at least one of: a higher stability or a higher level of expression when compared to a non-modified coronavirus spike protein, and a glycan shield similar to the virion. In one aspect, the method further comprising adding one or more adjuvants. In another aspect, the immunization is with the mutant coronavirus is a SARS-CoV-2 spike protein is selected from SEQ ID NOS:1 to 33. In another aspect, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages). In another aspect, the method further comprises isolating B cells from the immunized subject and obtaining the nucleic acid sequence of antibodies from the B cells, or fusing the isolated B cells with an immortalized cell to make a hybridoma. In another aspect, the mutant coronavirus spike proteins are formed into dimers, trimers, multimers, or nanoparticles. In another aspect, the nanoparticles comprise ferritin nanoparticles, polymeric nanoparticles, or both.

As embodied and broadly described herein, an alternative aspect of the present disclosure relates to a nucleic acid sequence encoding a mutant coronavirus spike protein comprising: one or more mutations that change an amino acid sequence of a coronavirus spike protein by at least one of: linking the S1/S2 subunits of a coronavirus spike protein, by deleting or removing a furin cleavage site loop and adding a short flexible peptide linker or a rigid peptide linker; adding at least one additional disulfide bond; or adding 1, 2, 3, 4, or 5 proline mutations for greater trimeric stability, wherein the resulting mutant coronavirus spike protein has at least one of: higher stability or level of expression, than a non-modified coronavirus spike protein, and a glycan shield similar to the virion. In another aspect, the mutant coronavirus is a SARS-CoV-2 spike protein is selected from SEQ ID NOS:1 to 33. In another aspect, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages). In another aspect, the mutant coronavirus spike proteins are formed into dimers, trimers, multimers, or nanoparticles. In another aspect, the nanoparticles comprise ferritin nanoparticles, polymeric nanoparticles, or both.

As embodied and broadly described herein, an alternative aspect of the present disclosure relates to a vector comprising a nucleic acid sequence encoding a mutant coronavirus spike protein comprising: one or more mutations that change the amino acid sequence by at least one of: linking the S1/S2 subunits of a coronavirus spike protein, by deleting a furin cleavage site loop and adding a short flexible peptide linker or a rigid peptide linker; adding at least one additional disulfide bond; or adding 1, 2, 3, 4, or 5 proline mutations for greater trimeric stability, wherein the resulting mutant coronavirus spike protein has at least one of: higher stability or level of expression, than a non-modified coronavirus spike protein. In another aspect, the vector is selected for expression in a bacteria, fungi, mammalian cell, avian cell, insect cell, or plant cell. In another aspect, the vector is in a bacteria, fungi, mammalian cell, avian cell, insect cell, or plant cell. In another aspect, the mutant coronavirus is a SARS-CoV-2 spike protein is selected from SEQ ID NOS:1 to 33. In another aspect, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages). In another aspect, the mutant coronavirus spike proteins are formed into dimers, trimers, multimers, or nanoparticles. In another aspect, the nanoparticles comprise ferritin nanoparticles, polymeric nanoparticles, or both.

### BRIEF DESCRIPTION OF THE DRAWINGS

For a more complete understanding of the features and advantages of the present invention, reference is now made to the detailed description of the invention along with the accompanying figures and in which:

FIG. 1 shows a construct of the present disclosure and size exclusion chromatography data from a redesigned furin cleavage site with flexible and/or rigid linkers.

FIG. 2 shows a construct of the present disclosure, size exclusion chromatography data, and relative expression data from the use of flexible and/or rigid linkers and proline modifications.

FIG. 3 shows protein expression gels with or without glutaraldehyde for the various constructs of the present disclosure, with the modifications listed in Table 1.

FIG. 4 is a 3-dimensional model of the SARS-CoV-2 spike protein trimer and the location of the various mutations of the present disclosure, as listed in Table 2.

FIG. 5 is a gel that shows the relative expression of the various constructs of the present disclosure.

FIG. 6 shows the expression data, dynamic light scattering data, and 3-dimensional location of the various mutants of the present disclosure.

FIG. 7 shows the dynamic light scattering data and gel showing the expression of the various mutants of the present disclosure.

FIG. 8 shows two constructs of the present disclosure (tetraproline and pentaproline), dynamic light scattering data and size exclusion chromatography data.

FIG. 9A shows a construct for a (V-five) Flexibly-Linked, Inter-Protomer spike (VFLIP).

FIG. 9B is a graph that shows differential scanning calorimetry for different constructs. FIG. 9C is a graph that shows the stability of the two different constructs under different conditions (4° C., lyophilized, 37° C. and 10× freeze/thaw cycles).

FIG. 10 shows that VFLIP also possesses a well-formed disulfide bond connecting two adjacent protomers that may result in more faithful display of quaternary epitopes.

FIG. 11 is a graph that shows neutralizing antibodies from mice immunized with the 5 different constructs as follows: (1) Parental S-2P, (2) HexaPro, (3) VFLIP, (4) VFLIP.D614G and (5) VFLIPΔFoldon adjuvanted with CpG+alum and boosted with the same four weeks later.

FIGS. 12A to 12C show: FIG. 12A shows that VFLIP is more thermostable than HexaPro, with 3° C. higher Tm. FIG. 12B shows that VFLIP retains its trimeric structure even after removal of the Foldon trimerization domain (VFLIPΔFoldon). FIG. 12C shows that VFLIPΔFoldon remains trimeric after lyophilization, multiple freeze/thaw cycles, and prolonged storage at either 4° C. or at room temperature.

FIGS. 13A to 13C show: FIG. 13A the immunization schedule and dosage, and assays using authentic D614G (FIG. 13B) and B.1.351 (FIG. 13C) SARS-CoV-2 showed that VFLIP-induced sera had a higher neutralizing potency compared to S-2P, with 50% neutralization at dilutions of 1:30,000 and 1:13,000, respectively.

FIGS. 14A and 14B show: FIG. 14A shows the immunization strategy, and FIG. 14B results from mice in all four groups, which mounted robust antibody responses as evidenced by total anti-spike antibody titers.

FIG. 15 shows graphs that show pseudovirus neutralization titers for VFLIP-immunized sera were significantly higher than S-2P and achieved 50% neutralization at dilutions over 1:100,000 in the samples collected one months after the second dose.

FIG. 16 shows graphs that show the results from assays using authentic D614G and B.1.351 variants which showed that VFLIP-induced sera had a higher neutralizing potency compared to S-2P, with 50% neutralization at dilutions of 1:30,000 and 1:13,000, respectively.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Structure-based vaccine design and in particular, iterative optimization of antigen has been a method successfully used in the last decade to achieve better vaccine candidates. Previously published initial designs to improve SARS-CoV-2 introduced two proline residues to stabilize the prefusion conformation, termed SARS S 2P. A second-generation SARS-CoV-2 S vaccine antigen termed HexaPro, also previously described, further stabilizes the antigen by introduction of four more proline residues.

The present disclosure describes a third-generation spike antigen, improved through iterative cycles of rational structure-based design that significantly increased both the transient expression yield of the antigen as well as its stability in different physical conditions. The resulting-third generation ‘USEO_DS’ stabilized immunogens contain one or more of three improvements over the current state-of-the-art HexaPro: (1) have their S1/S2 subunits genetically linked by replacement of their furin cleavage site loops by short flexible or rigid linkers, (2) their interprotomeric movements stabilized by an additional introduced disulfide bond, and (3) deletion of one of the six prolines in HexaPro (yielding PentaPro, but also 1, 2, 3 or 4 changes to or from proline) for greater trimeric pre-fusion stability. These USEO_DS immunogens maintain the structural characteristics corresponding to an uncleaved prefusion-stabilized S glycoprotein with a substantial improvement in the stability of the trimer against inactivation by heat, by freeze/thaw cycles and lyophilization/resuspension of the protein.

Vaccines currently deployed in the United States use a derivative of the prototypical, first-generation “S-2P” spike design (Pallesen et al. 2017), which contains two proline substitutions at positions 986 and 987 (Polack et al. 2020; Bos et al. 2020; Corbett et al. 2020; Wrapp et al. 2020). These vaccines have shown high efficacy in the short term, but the rapid timeframe for development has afforded few opportunities for antigen optimization. Recent work by Hsieh et al. illustrated that the S-2P spike exhibits relatively low yield and unfavorable purity (Hsieh et al. 2020). The poor yield may impact cost and manufacturability of vaccine candidates, and limit expression levels in vaccinated individuals, which in turn could necessitate higher doses and potentially increased reactogenicity. Moreover, several studies reported that S-2P protein preparations exhibit sensitivity to cold-temperature storage (Edwards et al. 2020; Xiong et al. 2020). Edwards et al. used negative-stain electron microscopy (NSEM) to demonstrate a 95% loss of well-formed S-2P spike trimers after 5-7 days of storage at 4° C. Exposure to 4° C. temperatures also resulted in lower thermostability and altered binding to monoclonal antibody (mAb) CR3022, suggesting perturbed structure and antigenicity.

A second-generation spike construct, termed “HexaPro”, contains four additional prolines at positions 817, 892, 899 and 942. HexaPro expresses to levels nearly 10-fold higher than those for wild-type spike or S-2P, has a 5° C. higher melting temperature (Tm) (Hsieh et al. 2020), and displays improved stability relative to S-2P under low-temperature storage and multiple freeze-thaw cycles (Edwards et al. 2020). Importantly, binding assays and cryoEM indicated that HexaPro better retains the native prefusion quaternary structure compared to S-2P, despite still exhibiting minor reductions in thermostability and mAb binding following incubation at 4° C.

Both S-2P and HexaPro, however, are prone to antibody- and ACE2-mediated separation or triggering of conformational change to the post-fusion state (Huo et al. 2020; Ge et al. 2021; Xiong et al. 2020). This triggering complicates structural analysis of mAb-spike and ACE2-spike complexes and may affect immunogenicity upon vaccination. Several spike constructs such as SR/X2 prevent this fusogenic activity with introduction of an inter-protomer disulfide bond, linking the RBD and the S2 subunits to “lock” the RBDs in the “down” conformation (Xiong et al. 2020; Henderson et al. 2020). Although these “locked-down” spike proteins maintain the trimeric state, the location of the inter-protomer disulfide bond prevents the natural hinge motion of the RBD and ablates binding to ACE2 and “RBD-up” antibodies, which are among the most potent neutralizers (Rogers et al. 2020; Liu et al. 2020; Huo et al. 2020; Brouwer et al. 2020). Furthermore, cryo-EM structures of a locked-down spike show that the RBDs are rotated 2A closer to the three-fold axis relative to wildtype (Xiong et al. 2020). These quaternary structure perturbations, together with locking of the RBD into an “all-down” state, could prevent elicitation and detection of protective antibodies against neutralizing epitopes that are only accessible in the “up” or mixed up/down conformation (Rogers et al. 2020; Huo et al. 2020; Liu et al. 2020; Brouwer et al. 2020). Even antibodies that target the “all-down” RBD conformation could be affected, particularly those that bridge two RBDs, such as the potent neutralizing mAbs S2M11, Nb6, and C144 (Schoof et al. 2020; Tortorici et al. 2020; Robbiani et al. 2020). Thus, a spike immunogen that preserves the natural RBD positioning and conformational dynamics is essential for maintaining the native antigenic landscape.

A central goal for SARS-CoV-2 vaccines is to reduce incidence of symptomatic disease through generation of enduring protective immunity. However, the recent emergence of SARS-CoV-2 variants of concern (VOC) poses a risk to first-generation vaccine efficacy and durability of both infection- and vaccine-induced humoral immunity. Lineage B.1.351 (informally known as the South African variant) is particularly concerning due to substitutions that confer increased transmissibility and reduced sensitivity to neutralization by heterotypic convalescent and vaccine-induced sera. Development of structurally designed vaccine candidates with improved immunogenicity and breadth of coverage is critical for controlling emergent VOC.

To address these issues associated with current spike constructs and emergence of VOC, the present inventors developed spike proteins containing different proline substitutions, cleavage site linkers, and interprotomer disulfide bonds. The present disclosure describes the production of “VFLIP” (five (V) prolines, Flexibly-Linked, Inter-Protomer disulfide) spikes that remain trimeric without exogenous trimerization motifs, and which have enhanced thermostability relative to earlier spike constructs. Surface plasmon resonance (SPR) and cryo-EM analysis confirm the native-like antigenicity of VFLIP and its improved utility for structural biology applications. Moreover, mice immunized with the VFLIP spike elicited significantly more potent neutralizing antibody responses against live SARS-CoV-2 D614G and B.1.351 compared to those immunized with S-2P. Taken together, the data demonstrate that VFLIP is a thermostable, covalently-linked, native-like spike trimer that represents a next-generation research reagent, diagnostic tool, immunogen, and vaccine.

As used herein, the term “antigen” refers to a mutant SARS-CoV-2 spike protein containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “adjuvant” refers to a substance that non-specifically changes or enhances an antigen-specific immune response of an organism to the antigen. Generally, adjuvants are non-toxic, have high-purity, are degradable, and are stable. With respect to the present disclosure, an adjuvant may be selected from aluminum hydroxide or mineral oil, and a stimulator of immune responses, such as Bordatella pertussis or Mycobacterium tuberculosis derived proteins. Suitable adjuvants are commercially available as, for example, Freund's Incomplete Adjuvant and Complete Adjuvant (Pifco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes; biodegradable microspheres; and Quil A. Suitable adjuvants also include, but are not limited to, toll-like receptor (TLR) agonists, particularly toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, and squalene. In some embodiments, the adjuvants are not bacterially-derived exotoxins. In an embodiment, adjuvants may include adjuvants which stimulate a Thl type response such as 3DMPL or QS21. Adjuvants may also include certain synthetic polymers such as poly amino acids and co-polymers of amino acids, saponin, paraffin oil, and muramyl dipeptide. Adjuvants also encompass genetic adjuvants such as immunomodulatory molecules encoded in a co-inoculated DNA, or as CpG oligonucleotides. The co-inoculated DNA can be in the same plasmid construct as the plasmid immunogen or in a separate DNA vector. The reader can refer to Vaccines (Basel). 2015 June; 3(2): 320-343 for further examples of suitable adjuvants.

As used herein, the term “immunological response” refers to an immune response to an antigen or composition that triggers in a subject a humoral and/or a cellular immune response to a mutant SARS-CoV-2 spike protein of the present disclosure. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art. In many instances, it will be desirable to have multiple administrations of the vaccine, usually not exceeding six to ten immunizations, more usually not exceeding four immunizations, e.g., one or more, usually at least about three immunizations. The immunizations will normally be at from two to twelve-week intervals, more usually from three to five week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as U.S. Pat. Nos. 3,791,932; 4,174,384 and 3,949,064, as illustrative of these types of assays.

The present disclosure can be used to generate one or more diagnostic and/or therapeutic antibodies against the novel antigens of the present disclosure. The antibodies can include polyclonal antibodies, such as those from immunized animals, but also include monoclonal antibodies made in vitro or in vivo. Both the polyclonal and monoclonal antibodies can be used in, e.g., radioimmunoassays, enzyme-linked immunosorbent assays, immunocytopathology, and flow cytometry for in vitro diagnosis, and in vivo for diagnosis and immunotherapy of human disease. Both the pan-specific and/or monoclonal antibodies of the present disclosure can be used for diagnosis and/or therapy of COVID19. Monoclonal antibodies may be generated by immunizing an animal, such as a mouse, isolating B cells from the immunized animal and fusing them with immortalized cells, as described by, e.g., Kohler and Milstein (1975, Nature 256:495-497), or as described by Kozbor et al. (1983, Immunology Today 4:72), or Cole et al. (1985 in Monoclonal Antibodies and Cancer Therapy, Alan R. Liss, Inc., pp. 77-96), relevant portions incorporated herein by reference. Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937), relevant portions incorporated herein by reference. For human use, the complementarity determining regions (CDRs) of the light and heavy chains of the monoclonal antibody can be engineered into a human antibody backbone or framework to make humanized antibodies.

In an aspect of the present disclosure is provided a method of diagnosing a coronavirus infection in a subject. In certain aspects, the method includes: (a) contacting a biological sample obtained from the subject with the mutant coronavirus spike protein provided herein including embodiments thereof, and (b) detecting binding of one or more antibodies to said mutant coronavirus spike protein, thereby diagnosing the coronavirus infection in said subject. In certain embodiments, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages).

In an aspect of the present disclosure is provided a method of diagnosing a SARS-CoV-2 infection in a subject. In certain aspects, the method includes: (a) contacting a biological sample obtained from the subject with the mutant coronavirus spike protein provided herein including embodiments thereof, and (b) detecting binding of one or more antibodies to said mutant coronavirus spike protein, thereby diagnosing the SARS-CoV-2 infection in said subject.

In an aspect of the present disclosure is provided a method for evaluating effectiveness of a coronavirus vaccine in a subject. In certain aspects, the method comprises (a) contacting a biological sample from a subject who has been administered with a vaccine for a coronavirus with the mutant coronavirus spike protein described herein, (b) detecting antibodies in the biological sample that specifically bind to the mutant coronavirus spike protein, and (c) performing quantitative and qualitative analysis of the antibodies detected in the biological sample, thereby evaluating effectiveness of the coronavirus vaccine in the subject. In certain embodiments, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages).

In an aspect of the present disclosure is provided a method for evaluating effectiveness of a SARS-CoV-2 vaccine in a subject. In certain aspects, the method comprises (a) contacting a biological sample from a subject who has been administered with a vaccine for a coronavirus with the mutant coronavirus spike protein described herein, (b) detecting antibodies in the biological sample that specifically bind to the mutant coronavirus spike protein, and (c) performing quantitative and qualitative analysis of the antibodies detected in the biological sample, thereby evaluating effectiveness of the SARS-CoV-2 vaccine in the subject.

As used herein, the term an “immunogenic composition” and “vaccine” refer to a composition that comprises a mutant SARS-CoV-2 spike protein, or a nucleic acid that expresses the mutant SARS-CoV-2 spike protein, where administration of the immunogenic composition or vaccine to a subject results in the development in the subject of a humoral and/or a cellular immune response to the antigenic molecule of interest, and by extension, to the virus.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

As used herein, the term a “coding sequence” or a sequence which “encodes” a mutant SARS-

CoV-2 spike polypeptide, refers to a nucleic acid molecule that is transcribed (in the case of DNA) and translated (in the case of mRNA) into a polypeptide when placed under the control of appropriate regulatory sequences (or “control elements”) and in vitro or in vivo. The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

As used herein, the term “control elements”, includes, but is not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences, and/or sequence elements controlling an open chromatin structure.

As used herein, “nanoparticles” refer to any particles, which are between 1 and 100 nanometers in size. The present disclosure includes formulations comprising the mutant coronavirus spike proteins of the present disclosure formed into nanoparticles or microparticles. In one example, nanoparticles or microparticles are formed with a protein and/or into a polymer matrix. The polymer matrix can be made with, e.g., poly (L-glycolic acid) (PLGA), polyglycolic acid (PGA), polylactic acid (PLA), poly(L-lactic acid) (PLLA), poly(epsilon-Caprolactone) PCL, Poly(methyl vinyl ether-co-maleic anhydride), polyglycolide, poly-L-lactide, poly-D-lactide, poly(amino acids), polyethyleneglycol PEG), polydioxanone, polycaprolactone, polygluconate, polylactic acid-polyethylene oxide copolymers, polyorthoesters, polyhydroxybutyrate, polyanhydride, polyphosphoester, poly(alpha-hydroxy acid), ferritin, chitosan, alginate, collagen, dextran, polyester, cellulose, carboxymethyl cellulose, modified cellulose, collagen, or combinations thereof. In some examples, the nanoparticles are partially or fully biodegradable.

As used herein, the term “nucleic acid” includes, but is not limited to, DNA or RNA that encodes the mutant SARS-CoV-2 spike proteins of the present disclosure, whether expressed or optimized for prokaryotic or eukaryotic expression. The term also captures sequences that include any of the known base analogs of DNA and RNA.

As used herein, the term “operably linked” refers to an arrangement of elements wherein the components so described are configured so as to perform their usual function. Thus, a given promoter operably linked to a coding sequence is capable of effecting the expression of the coding sequence when active. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

As used herein, the term “recombinant” refers to a polynucleotide that encodes the mutant SARS-CoV-2 spike protein whether from the viral genome, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting prokaryotic microorganisms or eukaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

Techniques for determining amino acid sequence “similarity” are well known in the art. In general, “similarity” means the exact amino acid to amino acid comparison of two or more polypeptides at the appropriate place, where amino acids are identical or possess similar chemical and/or physical properties such as charge or hydrophobicity. A so-termed “percent similarity” then can be determined between the compared polypeptide sequences. Techniques for determining nucleic acid and amino acid sequence identity also are well known in the art and include determining the nucleotide sequence of the mRNA for that gene (usually via a cDNA intermediate) and determining the amino acid sequence encoded thereby and comparing this to a second amino acid sequence. In general, “identity” refers to an exact nucleotide to nucleotide or amino acid to amino acid correspondence of two polynucleotides or polypeptide sequences, respectively.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “mutant coronavirus spike protein” or “VFLIP” as provided herein includes any of the recombinant or naturally-occurring forms of a coronavirus spike protein, or variants or homologs thereof that maintain coronavirus Spike protein activity (e.g. within at least 50%, 80%, 90%, 95%, 96%, 97%, 98%, 99% or 100% activity compared to coronavirus Spike Protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring coronavirus Spike protein polypeptide. In embodiments, coronavirus Spike protein is the protein as identified by the UniProt reference number PODTC2, or a variant, homolog or functional fragment thereof. In aspects, the mutant coronavirus spike protein includes the amino acid sequence of one of SEQ ID NOs:1-33. In aspects, the mutant coronavirus spike protein has the amino acid sequence of one of SEQ ID NOs:1-33.

Two or more polynucleotide sequences can be compared by determining their “percent identity.” Two or more amino acid sequences likewise can be compared by determining their “percent identity.” The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. 0. Dayhoff ed., 5 suppl. 3:353-358, National Biomedical Research Foundation, Washington, D.C., USA, and normalized by Gribskov, Nucl. Acids Res. 14(6):6745-6763 (1986), relevant portion incorporated herein by reference. Suitable programs for calculating the percent identity or similarity between sequences are generally known in the art.

As used herein, the term a “vector” refers to a nucleic acid capable of transferring gene sequences to target cells (e.g., bacterial plasmid vectors, viral vectors, non-viral vectors, particulate carriers, and liposomes). Typically, “vector construct,” “expression vector,” and “gene transfer vector,” mean any nucleic acid construct capable of directing the expression of one or more sequences of interest in a host cell. Thus, the term includes cloning and expression vehicles, as well as viral vectors. The term is used interchangeable with the terms “nucleic acid expression vector” and “expression cassette.”

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portions of any of the above are incorporated herein by reference.

As used herein, the term “subject” refers to any member of the subphylum chordata, including, but not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

As used herein, the term “administering” refers to oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

As used herein, the term “co-administer” refers to a compound or composition described herein that is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols. The preparations may also be combined with inhaled mucolytics (e.g., rhDNase, as known in the art) or with inhaled bronchodilators (short or long acting beta agonists, short or long acting anticholinergics), inhaled corticosteroids, or inhaled antibiotics to improve the efficacy of these drugs by providing additive or synergistic effects. The compositions of the present invention can be delivered transdermally, by a topical route, formulated as applicator sticks, solutions, suspensions, emulsions, gels, creams, ointments, nanoparticles, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles, such as protein nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer or infection in a subject who has been diagnosed with the cancer or infection). The administration of vaccines is referred to vaccination. In embodiments, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus).

Pharmaceutical compositions can also include large, slowly metabolized macromolecules such as proteins, polysaccharides such as chitosan, polylactic acids, polyglycolic acids and copolymers (such as latex functionalized sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

As used herein, the term “treatment” refers to any of (i) the prevention of infection or reinfection with SARS-CoV-2, as in a traditional vaccine, (ii) the reduction or elimination of symptoms, and (iii) the substantial or complete elimination of the pathogen in question. Treatment may be effected prophylactically (prior to infection) or therapeutically (following infection).

As used herein, the term “effective dose” refers to that amount of one or more mutant SARS-CoV-2 spike proteins of the disclosure sufficient to induce immunity, to prevent and/or ameliorate an infection or to reduce at least one symptom of an infection and/or to enhance the efficacy of another dose of a SARS-CoV-2. An effective dose may refer to the amount of a mutant SARS-CoV-2 spike protein sufficient to delay or minimize the onset of an infection. An effective dose may also refer to the amount of a mutant SARS-CoV-2 spike protein that provides a therapeutic benefit in the treatment or management of an infection. Further, an effective dose is the amount with respect to a mutant SARS-CoV-2 spike protein of the disclosure alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as the mutant SARS-CoV-2 spike proteins of the disclosure or can be administered separately. Either the protein or an expression vector encoding the protein can be administered to produce an immunostimulatory effect.

As used herein, the term “protective immune response” or “protective response” refers to an immune response mediated by antibodies against an infectious agent, which is exhibited by a vertebrate (e.g., a human), which prevents or ameliorates an infection or reduces at least one symptom thereof. Mutant SARS-CoV-2 spike proteins of the disclosure can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates flavivirus infection or reduces at least one symptom thereof.

As used herein, the term “antigenic formulation” or “antigenic composition” refers to a

preparation which, when administered to a vertebrate, e.g., a mammal, will induce an immune response.

As used herein, the terms “immunization” or “vaccine” are used interchangeably to refer to a formulation which contains one or more of the mutant SARS-CoV-2 spike proteins of the present disclosure, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of the mutant SARS-CoV-2 spike proteins. Typically, the vaccine comprises a conventional saline or buffered aqueous solution medium in which the composition of the present disclosure is suspended or dissolved. In this form, the composition of the present disclosure can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The practice of the present disclosure employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

### Example 1

### Structure-Based Design of a Highly Stable, Covalently-Linked SARS-Cov-2 Spike Trimer with Improved Structural Properties and Immunogenicity

Increase in the expression level of SARS2 Spike antigens by redesigning the furin cleavage site loop with flexible and rigid linkers. As a first step in the structure-based optimization of the SARS2 spike, the inventors hypothesized that the long and flexible cleavage site loop (amino acids 676-690), poorly conserved in related betacoronavirus and not yet visible in any high-resolution structure, could contribute to the destabilization of the protein fold. Therefore, replacing the amino acids in positions 676-690 by short linkers could improve the expression and the stability of the antigen without disturbing the overall antigenic architecture.

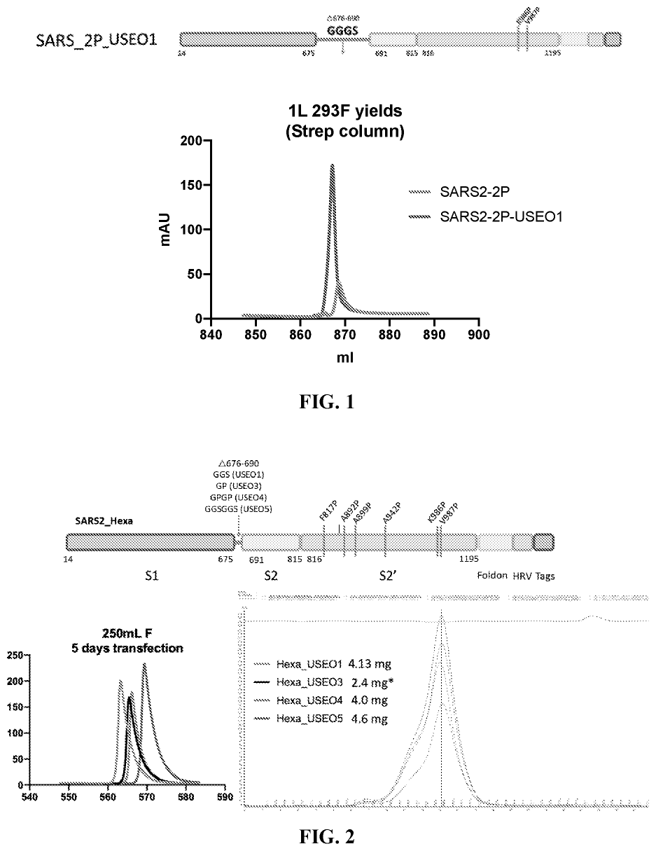

The resulting SARS2_S_2P proteins with the redesigned short linkers named SARS2_2_2P_USED expressed approximately 4-fold more than SARS2_S_2P. FIG. 1 shows a construct of the present disclosure and size exclusion chromatography data from a redesigned furin cleavage site with flexible and/or rigid linkers.

As a second step, second-generation versions of the known Hexapro SARS2 spike antigens were created by adding the linkers USEO1 (GGS) (SEQ ID NO: 34), USEO3 (GP) (SEQ ID NO: 35), USEO4 (GPGP)(SEQ ID NO:36) and USEOS (GGSGGS)(SEQ ID NO:37) to Hexapro SARS2 spike. After purification by affinity- and size-exclusion chromatography (SEC) the resulting proteins behave similarly to HexaPro in SEC profile and band distribution on SDS-PAGE under native conditions (Blue-native gel), or under reducing or non-reducing denaturing conditions (SDS-PAGE). All constructs Hexa_USEO1, Hexa_USEO3, Hexa_USEO4, and Hexa_USEOS yielded a trimeric state of the SARS-CoV2 protein.

FIG. 2 shows a construct of the present disclosure, size exclusion chromatography data, and relative expression data from the use of flexible and/or rigid linkers and proline modifications.

Those Hexa_USED-designs increase the expression level of the antigens over the previously published HexaPo Spike protein between 5-25% without disturbing the overall antigenicity of the proteins. Hexa_USED-proteins react equivalently to SARS-CoV2 convalescent sera mAb CR3022 in ELISA binding assays.

TABLE 1
Linkers that replace the furin cleavage site loop.

| | | | % increased yield |
|---|---|---|---|
| Construct name | Deletion | Insertion | (compared to HexaPro) |
| Hexa_USEO1 | 676-690 | GGS | 15% |
| Hexa_USEO3 | 676-690 | GP | 5% |
| Hexa_USEO4 | 676-690 | GPGP | 5% |
| Hexa_USEO5 | 676-690 | GGSGGS | 25% |

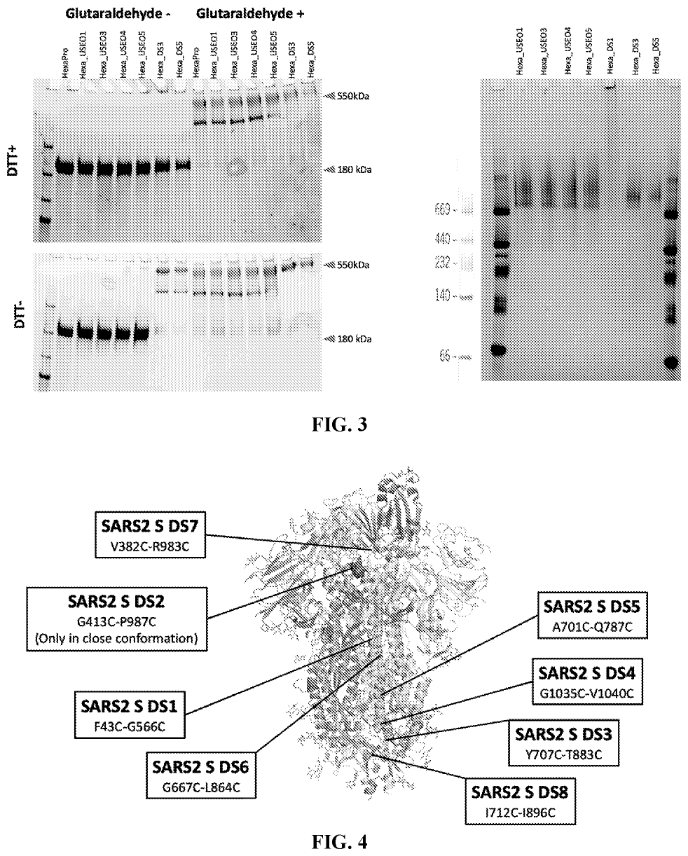

FIG. 3 shows protein expression gels with or without glutaraldehyde for the various constructs of the present disclosure, with the modifications listed in Table 1. GGS (SEQ ID NO:34), GP (SEQ ID NO:35), GPGP (SEQ ID NO:36), or GGSGGS (SEQ ID NO:37).

Increase in the stability of SARS-CoV-2 Spike protein through the covalent union of protomers through disulfide bounds.

Additional improves to stability were obtained by introduction of a novel disulfide bond. The inventors evaluated disulfide bond introductions first in the first-generation SARS2 S 2P protein framework. Eight different candidate disulfide bonds (constructs DS1-DS8) based on analysis of the structure of SARS-CoV-2 Spike were made and expressed individually by transient transfection in HEK293F cells.

TABLE 2
Additional disulfide bonds.

| Disulfide bound | Mutated amino acids | Chains linked |
|---|---|---|
| DS1 | F43C-G566C | S1-S1 |
| DS2 | G413C-P987C | S1-S2′ |
| DS3 | Y707C-T883C | S2-S2′ |
| DS4 | G1035C-V1040C | S2′-S2′ |
| DS5 | A701C-Q787C | S2′-S2′ |
| DS6 | G667C-L864C | S1-S2′ |
| DS7 | V382C-R983C | S1-S2′ |
| DS8 | I712C-I816C | S2-S2′ |

FIG. 4 is a 3-dimensional model of the SARS-CoV-2 spike protein trimer and the location of the various mutations of the present disclosure, as listed in Table 2.

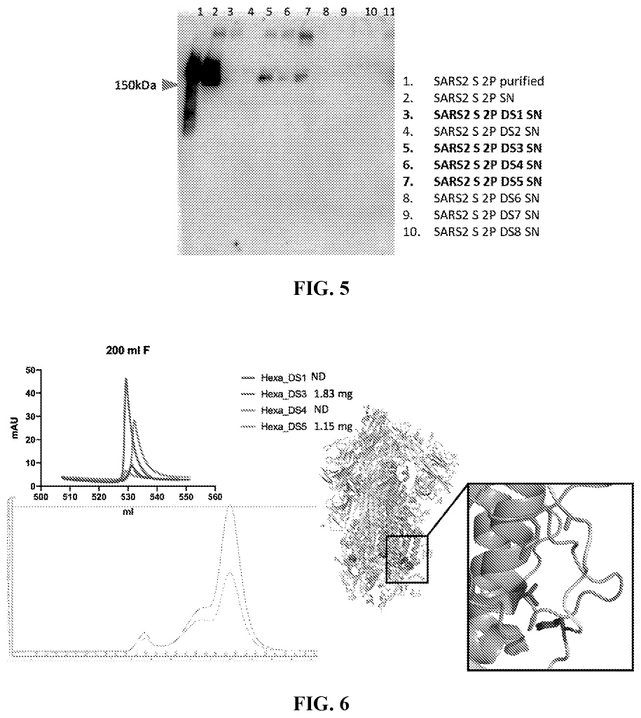

The supernatants were collected, and a Western Blot was carried out, with detection via a polyclonal antibody against the Streptavidin purification tag. The result of the Western Blot shows that the proteins with the disulfide bonds DS1, DS3, DS4, and DS5 were expressed, although in a lower proportion than the control SARS2 S-2P. FIG. 5 is a gel that shows the relative expression of the various constructs of the present disclosure.

Next, DS1, DS3, DS4, and DS5 were next cloned in the second-generation SARS2_S_HexaPro backbone. The resulting proteins, Hexa DS1-5, contain the novel disulfide, six introduced prolines, but not the introduced linker between Si and S2 subunits. Hexa DS1-5 were transiently expressed in HEK293F cells and purified by affinity chromatography in Streptactin columns. Two candidates, Hexa DS3 and Hexa DSS, were found to express with better yield than the others. Both Hexa DS3 and Hexa DS5 yielded perfectly formed trimers, as evidenced by in SEC, as well as in SDS-PAGE under denaturing and reducing conditions after cross-linking with glutaraldehyde.

Finally, the stability of the trimers was analyzed by Dynamic Light Scattering (DLS), by lyophilization of the proteins, by analysis at a range of temperatures, and visualization of the treated proteins in SDS-PAGE with glutaraldehyde.

FIG. 6 shows the expression data, dynamic light scattering data, and 3-dimensional location of the various mutants of the present disclosure. The results of the DLS show that, while all the proteins analyzed have the same pattern of light scattering in a temperature ramp, the proteins with the disulfide bond DS3 (Hexa DS3) are more stably folded.

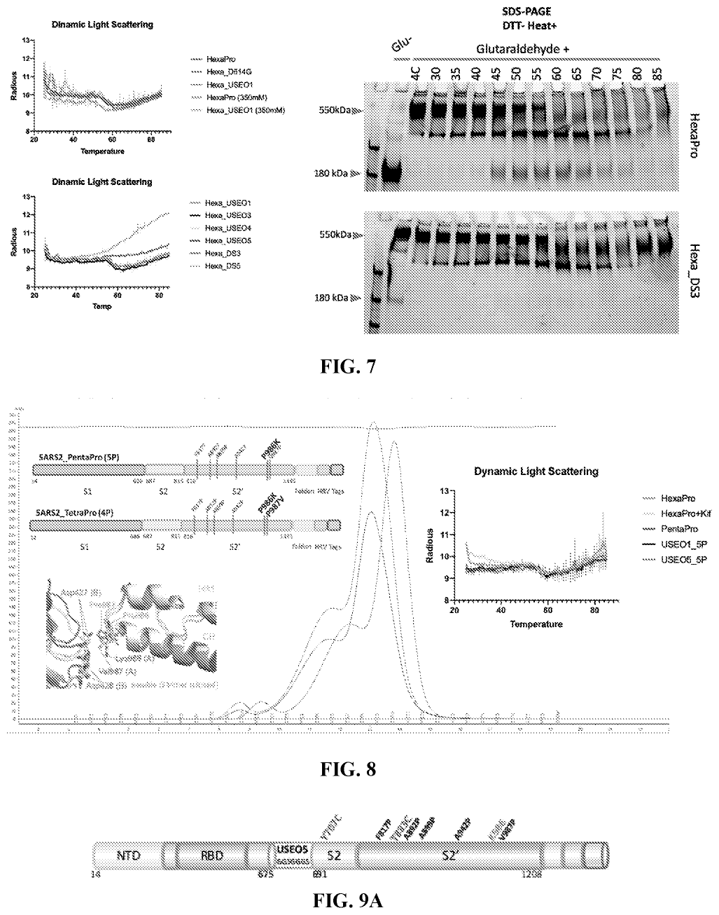

In gels where proteins were heated for 10 minutes and then cross-linked with glutaraldehyde and loaded SDS-PAGE gels, second-generation HexaPro begins to lose its trimeric structure at 45° C. (the band corresponding to 550 kDa begins to be less visible and a band of 180 kDa appears). In contrast, third-generation spike containing DS3 disulfide bond (Hexa_DS3) remains stable as a 550 kDa trimer up to approximately 55° C. FIG. 7 shows the dynamic light scattering data and gel showing the expression of the various mutants of the present disclosure.

Conversion from HexaPro to PentaPro to mimic the SARS2 Spike protein with the wildtype.

The structural analysis of the first-generation SARS2 Spike protein, SARS2_S_2P, which was stabilized by the K986P and V987P mutations, suggests that the K986P mutation is probably disrupting a salt bridge between K986 and an aspartic acid located at positions D427 or D428 of the adjacent monomer. This loss of a salt bridge by the first of the two-Pro, although stabilizing an individual monomer in its prefusion state, may destabilize the trimeric assembly of the three monomers. The inventors hypothesized that reverting the mutation (P986K) to five prolines instead of six will make the protein more stable without losing its pre-fusion condition. In one example, the 1, 2, 3, 4, or 5 modifications can be selected from: F817P, A892P, A899P, A942P, P986K, K986P, V987P, and P987V, and preferably, F817P, A892P, A899P, A942P, K986P, and V987P.

In this third step in optimization, PentaPro molecules were designed that also include the redesigned cleavage site loop (USEO1-5) and five prolines termed USE0(1-5)_5P that consistently express better than their corresponding backbone protein and that equal or exceed the stability of the trimer measured by DLS.

New versions containing all three types of modifications (linker, PentaPro, and novel disulfide) can be used with the present disclosure.

FIG. 8 shows two constructs of the present disclosure (tetraproline and pentaproline), dynamic light scattering data and size exclusion chromatography data.

Amino acid sequences for the
constructs listed above.
Hexa_USEO1
Linker underlined.
(SEQ ID NO: 1)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVFNATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGGSSIIAYTMSLGAENSV
AYSNNSIAIPTNFTISVTTEILPVSMTKTSVDCTMYICGD
STECSNLLLQYGSFCTQLNRALTGIAVEQDKNTQEVFAQV
KQIYKTPPIKDFGGFNFSQILPDPSKPSKRSPIEDLLFNK
VTLADAGFIKQYGDCLGDIAARDLICAQKFNGLTVLPPLL
TDEMIAQYTSALLAGTITSGWTFGAGPALQIPFPMQMAYR
FNGIGVTQNVLYENQKLIANQFNSAIGKIQDSLSSTPSAL
GKLQDVVNQNAQALNTLVKQLSSNFGAISSVLNDILSRLD
PPEAEVQIDRLITGRLQSLQTYVTQQLIRAAEIRASANLA
ATKMSECVLGQSKRVDFCGKGYHLMSFPQSAPHGVVFLHV
TYVPAQEKNFTTAPAICHDGKAHFPREGVFVSNGTHWFVT
QRNFYEPQIITTDNTFVSGNCDVVIGIVNNTVYDPLQPEL
DSFKEELDKYFKNHTSPDVDLGDISGINASVVNIQKEIDR
LNEVAKNLNESLIDLQELGKYEQ
Hexa_USEO3
Linker underlined
(SEQ ID NO: 2)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGFNCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGPSIIAYTMSLGAENSVA
YSNNSIAIPTNFTISVTTEILPVSMTKTSVDCTMYICGDS
TECSNLLLQYGSFCTQLNRALTGIAVEQDKNTQEVFAQVK
QIYKTPPIKDFGGFNFSQILPDPSKPSKRSPIEDLLENKV
TLADAGFIKQYGDCLGDIAARDLICAQKFNGLTVLPPLLT
DEMIAQYTSALLAGTITSGWTFGAGPALQIPFPMQMAYRF
NGIGVTQNVLYENQKLIANQFNSAIGKIQDSLSSTPSALG
KLQDVVNQNAQALNTLVKQLSSNFGAISSVLNDILSRLDP
PEAEVQIDRLITGRLQSLQTYVTQQLIRAAEIRASANLAA
TKMSECVLGQSKRVDFCGKGYHLMSFPQSAPHGVVFLHVT
YVPAQEKNFTTAPAICHDGKAHFPREGVFVSNGTHWFVTQ
RNFYEPQIITTDNTFVSGNCDVVIGIVNNTVYDPLQPELD
SFKEELDKYFKNHTSPDVDLGDISGINASVVNIQKEIDRL
NEVAKNLNESLIDLQELGKYEQ
Hexa_USEO4
Linker underlined
(SEQ ID NO: 3)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGPGPSIIAYTMSLGAENS
VAYSNNSIAIPTNFTISVTTEILPVSMTKTSVDCTMYICG
DSTECSNLLLQYGSFCTQLNRALTGIAVEQDKNTQEVFAQ
VKQIYKTPPIKDFGGFNFSQILPDPSKPSKRSPIEDLLFN
KVTLADAGFIKQYGDCLGDIAARDLICAQKFNGLTVLPPL
LTDEMIAQYTSALLAGTITSGWTFGAGPALQIPFPMQMAY
RENGIGVTQNVLYENQKLIANQFNSAIGKIQDSLSSTPSA
LGKLQDVVNQNAQALNTLVKQLSSNFGAISSVLNDILSRL
DPPEAEVQIDRLITGRLQSLQTYVTQQLIRAAEIRASANL
AATKMSECVLGQSKRVDFCGKGYHLMSFPQSAPHGVVFLH
VTYVPAQEKNFTTAPAICHDGKAHFPREGVFVSNGTHWFV
TQRNFYEPQIITTDNTFVSGNCDVVIGIVNNTVYDPLQPE
LDSFKEELDKYFKNHTSPDVDLGDISGINASVVNIQKEID
RLNEVAKNLNESLIDLQELGKYEQ
Hexa_USEO5
Linker underlined
(SEQ ID NO: 4)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGGSGGSSIIAYTMSLGAE
NSVAYSNNSIAIPTNFTISVTTEILPVSMTKTSVDCTMYI
CGDSTECSNLLLQYGSFCTQLNRALTGIAVEQDKNTQEVF
AQVKQIYKTPPIKDFGGFNFSQILPDPSKPSKRSPIEDLL
ENKVTLADAGFIKQYGDCLGDIAARDLICAQKFNGLTVLP
PLLTDEMIAQYTSALLAGTITSGWTFGAGPALQIPFPMQM
AYRENGIGVTQNVLYENQKLIANQFNSAIGKIQDSLSSTP
SALGKLQDVVNQNAQALNTLVKQLSSNFGAISSVLNDILS
RLDPPEAEVQIDRLITGRLQSLQTYVTQQLIRAAEIRASA
NLAATKMSECVLGQSKRVDFCGKGYHLMSFPQSAPHGVVF
LHVTYVPAQEKNFTTAPAICHDGKAHFPREGVFVSNGTHW
FVTQRNFYEPQIITTDNTFVSGNCDVVIGIVNNTVYDPLQ
PELDSFKEELDKYFKNHTSPDVDLGDISGINASVVNIQKE
IDRLNEVAKNLNESLIDLQELGKYEQ
Hexa_DS1
Cysteine modifications bolded
(SEQ ID NO: 5)
QCVNLTTRTQLPPAYTNSFTRGVYYPDKVCRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFCRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQTQTNSPGSASSVASQSII
AYTMSLGAENSVAYSNNSIAIPTNFTISVTTEILPVSMTK
TSVDCTMYICGDSTECSNLLLQYGSFCTQLNRALTGIAVE
QDKNTQEVFAQVKQIYKTPPIKDFGGFNFSQILPDPSKPS
KRSPIEDLLFNKVTLADAGFIKQYGDCLGDIAARDLICAQ
KFNGLTVLPPLLTDEMIAQYTSALLAGTITSGWTFGAGPA
LQIPFPMQMAYRENGIGVTQNVLYENQKLIANQFNSAIGK
IQDSLSSTPSALGKLQDVVNQNAQALNTLVKQLSSNFGAI
SSVLNDILSRLDPPEAEVQIDRLITGRLQSLQTYVTQQLI
RAAEIRASANLAATKMSECVLGQSKRVDFCGKGYHLMSFP
QSAPHGVVFLHVTYVPAQEKNFTTAPAICHDGKAHFPREG
VFVSNGTHWFVTQRNFYEPQIITTDNTFVSGNCDVVIGIV
NNTVYDPLQPELDSFKEELDKYFKNHTSPDVDLGDISGIN
ASVVNIQKEIDRLNEVAKNLNESLIDLQELGKYEQ
Hexa_DS3
Cysteine modifications bolded
(SEQ ID NO: 6)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQTQTNSPGSASSVASQSII
AYTMSLGAENSVACSNNSIAIPTNFTISVTTEILPVSMTK
TSVDCTMYICGDSTECSNLLLQYGSFCTQLNRALTGIAVE
QDKNTQEVFAQVKQIYKTPPIKDFGGFNFSQILPDPSKPS
KRSPIEDLLFNKVTLADAGFIKQYGDCLGDIAARDLICAQ
KFNGLTVLPPLLTDEMIAQYTSALLAGTICSGWTFGAGPA
LQIPFPMQMAYRENGIGVTQNVLYENQKLIANQFNSAIGK
IQDSLSSTPSALGKLQDVVNQNAQALNTLVKQLSSNFGAI
SSVLNDILSRLDPPEAEVQIDRLITGRLQSLQTYVTQQLI
RAAEIRASANLAATKMSECVLGQSKRVDFCGKGYHLMSFP
QSAPHGVVFLHVTYVPAQEKNFTTAPAICHDGKAHFPREG
VFVSNGTHWFVTQRNFYEPQIITTDNTFVSGNCDVVIGIV
NNTVYDPLQPELDSFKEELDKYFKNHTSPDVDLGDISGIN
ASVVNIQKEIDRLNEVAKNLNESLIDLQELGKYEQ
Hexa_DS4
Cysteine modifications bolded
(SEQ ID NO: 7)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQTQTNSPGSASSVASQSII
AYTMSLGAENSVAYSNNSIAIPTNFTISVTTEILPVSMTK
TSVDCTMYICGDSTECSNLLLQYGSFCTQLNRALTGIAVE
QDKNTQEVFAQVKQIYKTPPIKDFGGFNFSQILPDPSKPS
KRSPIEDLLFNKVTLADAGFIKQYGDCLGDIAARDLICAQ
KFNGLTVLPPLLTDEMIAQYTSALLAGTITSGWTFGAGPA
LQIPFPMQMAYRFNGIGVTQNVLYENQKLIANQFNSAIGK
IQDSLSSTPSALGKLQDVVNQNAQALNTLVKQLSSNFGAI
SSVLNDILSRLDPPEAEVQIDRLITGRLQSLQTYVTQQLI
RAAEIRASANLAATKMSECVLCQSKRCDFCGKGYHLMSFP
QSAPHGVVFLHVTYVPAQEKNFTTAPAICHDGKAHFPREG
VFVSNGTHWFVTQRNFYEPQIITTDNTFVSGNCDVVIGIV
NNTVYDPLQPELDSFKEELDKYFKNHTSPDVDLGDISGIN
ASVVNIQKEIDRLNEVAKNLNESLIDLQELGKYEQ
Hexa_DS5
Cysteine modifications bolded
(SEQ ID NO: 8)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQTQTNSPGSASSVASQSII
AYTMSLGCENSVAYSNNSIAIPTNFTISVTTEILPVSMTK
TSVDCTMYICGDSTECSNLLLQYGSFCTQLNRALTGIAVE
QDKNTQEVFAQVKCIYKTPPIKDFGGFNFSQILPDPSKPS
KRSPIEDLLFNKVTLADAGFIKQYGDCLGDIAARDLICAQ
KFNGLTVLPPLLTDEMIAQYTSALLAGTITSGWTFGAGPA
LQIPFPMQMAYRFNGIGVTQNVLYENQKLIANQFNSAIGK
IQDSLSSTPSALGKLQDVVNQNAQALNTLVKQLSSNFGAI
SSVLNDILSRLDPPEAEVQIDRLITGRLQSLQTYVTQQLI
RAAEIRASANLAATKMSECVLGQSKRVDFCGKGYHLMSFP
QSAPHGVVFLHVTYVPAQEKNFTTAPAICHDGKAHFPREG
VFVSNGTHWFVTQRNFYEPQIITTDNTFVSGNCDVVIGIV
NNTVYDPLQPELDSFKEELDKYFKNHTSPDVDLGDISGIN
ASVVNIQKEIDRLNEVAKNLNESLIDLQELGKYEQ
PentaPro (5P)
proline modifications bold
underlined
(SEQ ID NO: 9)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVFNATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQTQTNSPGSASSVASQSII
AYTMSLGAENSVAYSNNSIAIPTNFTISVTTEILPVSMTK
TSVDCTMYICGDSTECSNLLLQYGSFCTQLNRALTGIAVE
QDKNTQEVFAQVKQIYKTPPIKDFGGFNFSQILPDPSKPS
KRSPIEDLLFNKVTLADAGFIKQYGDCLGDIAARDLICAQ
KFNGLTVLPPLLTDEMIAQYTSALLAGTITSGWTFGAGPA
LQIPFPMQMAYRFNGIGVTQNVLYENQKLIANQFNSAIGK
IQDSLSSTPSALGKLQDVVNQNAQALNTLVKQLSSNFGAI
SSVLNDILSRLDKPEAEVQIDRLITGRLQSLQTYVTQQLI
RAAEIRASANLAATKMSECVLGQSKRVDFCGKGYHLMSFP
QSAPHGVVFLHVTYVPAQEKNFTTAPAICHDGKAHFPREG
VFVSNGTHWFVTQRNFYEPQIITTDNTFVSGNCDVVIGIV
NNTVYDPLQPELDSFKEELDKYFKNHTSPDVDLGDISGIN
ASVVNIQKEIDRLNEVAKNLNESLIDLQELGKYEQ
5P_USEO1
linker underlined, proline
modifications bold underlined
(SEQ ID NO: 10)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGGSSIIAYTMSLGAENSV
AYSNNSIAIPTNFTISVTTEILPVSMTKTSVDCTMYICGD
STECSNLLLQYGSFCTQLNRALTGIAVEQDKNTQEVFAQV
KQIYKTPPIKDFGGFNFSQILPDPSKPSKRSPIEDLLFNK
VTLADAGFIKQYGDCLGDIAARDLICAQKFNGLTVLPPLL
TDEMIAQYTSALLAGTITSGWTFGAGPALQIPFPMQMAYR
FNGIGVTQNVLYENQKLIANQFNSAIGKIQDSLSSTPSAL
GKLQDVVNQNAQALNTLVKQLSSNFGAISSVLNDILSRLD
KPEAEVQIDRLITGRLQSLQTYVTQQLIRAAEIRASANLA
ATKMSECVLGQSKRVDFCGKGYHLMSFPQSAPHGVVFLHV
TYVPAQEKNFTTAPAICHDGKAHFPREGVFVSNGTHWFVT
QRNFYEPQIITTDNTFVSGNCDVVIGIVNNTVYDPLQPEL
DSFKEELDKYFKNHTSPDVDLGDISGINASVVNIQKEIDR
LNEVAKNLNESLIDLQELGKYEQ
5P_USEO3
linker underlined, proline modifications
bold underlined
(SEQ ID NO: 11)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGPSIIAYTMSLGAENSVA
YSNNSIAIPTNFTISVTTEILPVSMTKTSVDCTMYICGDS
TECSNLLLQYGSFCTQLNRALTGIAVEQDKNTQEVFAQVK
QIYKTPPIKDFGGFNFSQILPDPSKPSKRSPIEDLLFNKV
TLADAGFIKQYGDCLGDIAARDLICAQKFNGLTVLPPLLT
DEMIAQYTSALLAGTITSGWTFGAGPALQIPFPMQMAYRE
NGIGVTQNVLYENQKLIANQFNSAIGKIQDSLSSTPSALG
KLQDVVNQNAQALNTLVKQLSSNFGAISSVLNDILSRLDK
PEAEVQIDRLITGRLQSLQTYVTQQLIRAAEIRASANLAA
TKMSECVLGQSKRVDFCGKGYHLMSFPQSAPHGVVFLHVT
YVPAQEKNFTTAPAICHDGKAHFPREGVFVSNGTHWFVTQ
RNFYEPQIITTDNTFVSGNCDVVIGIVNNTVYDPLQPELD
SFKEELDKYFKNHTSPDVDLGDISGINASVVNIQKEIDRL
NEVAKNLNESLIDLQELGKYEQ
5P_USEO4
linker underlined, proline modifications
bold underlined
(SEQ ID NO: 12)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGPGPSIIAYTMSLGAENS
VAYSNNSIAIPTNFTISVTTEILPVSMTKTSVDCTMYICG
DSTECSNLLLQYGSFCTQLNRALTGIAVEQDKNTQEVFAQ
VKQIYKTPPIKDFGGFNFSQILPDPSKPSKRSPIEDLLFN
KVTLADAGFIKQYGDCLGDIAARDLICAQKFNGLTVLPPL
LTDEMIAQYTSALLAGTITSGWTFGAGPALQIPFPMQMAY
RFNGIGVTQNVLYENQKLIANQFNSAIGKIQDSLSSTPSA
LGKLQDVVNQNAQALNTLVKQLSSNFGAISSVLNDILSRL
DKPEAEVQIDRLITGRLQSLQTYVTQQLIRAAEIRASANL
AATKMSECVLGQSKRVDFCGKGYHLMSFPQSAPHGVVFLH
VTYVPAQEKNFTTAPAICHDGKAHFPREGVFVSNGTHWFV
TQRNFYEPQIITTDNTFVSGNCDVVIGIVNNTVYDPLQPE
LDSFKEELDKYFKNHTSPDVDLGDISGINASVVNIQKEID
RLNEVAKNLNESLIDLQELGKYEQ
5P_USEO5
linker underlined, proline modifications
bold underlined
(SEQ ID NO: 13)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGFNCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGGSGGSSSIIAYTMSLGA
ENSVAYSNNSIAIPTNFTISVTTEILPVSMTKTSVDCTMY
ICGDSTECSNLLLQYGSFCTQLNRALTGIAVEQDKNTQEV
FAQVKQIYKTPPIKDFGGFNFSQILPDPSKPSKRSPIEDL
LENKVTLADAGFIKQYGDCLGDIAARDLICAQKFNGLTVL
PPLLTDEMIAQYTSALLAGTITSGWTFGAGPALQIPFPMQ
MAYRFNGIGVTQNVLYENQKLIANQFNSAIGKIQDSLSST
PSALGKLQDVVNQNAQALNTLVKQLSSNFGAISSVLNDIL
SRLDKPEAEVQIDRLITGRLQSLQTYVTQQLIRAAEIRAS
ANLAATKMSECVLGQSKRVDFCGKGYHLMSFPQSAPHGVV
FLHVTYVPAQEKNFTTAPAICHDGKAHFPREGVFVSNGTH
WFVTQRNFYEPQIITTDNTFVSGNCDVVIGIVNNTVYDPL
QPELDSFKEELDKYFKNHTSPDVDLGDISGINASVVNIQK
EIDRLNEVAKNLNESLIDLQELGKYEQ
USEO5_5P_DS3
linker underlined, cysteine bolded,
proline modifications
bold underlined is a VLIP
(SEQ ID NO: 14)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGGSGGSSIIAYTMSLGAE
NSVACSNNSIAIPTNFTISVTTEILPVSMTKTSVDCTMYI
CGDSTECSNLLLQYGSFCTQLNRALTGIAVEQDKNTQEVF
AQVKQIYKTPPIKDFGGFNFSQILPDPSKPSKRSPIEDLL
ENKVTLADAGFIKQYGDCLGDIAARDLICAQKFNGLTVLP
PLLTDEMIAQYTSALLAGTICSGWTFGAGPALQIPFPMQM
AYRENGIGVTQNVLYENQKLIANQFNSAIGKIQDSLSSTP
SALGKLQDVVNQNAQALNTLVKQLSSNFGAISSVLNDILS
RLDKPEAEVQIDRLITGRLQSLQTYVTQQLIRAAEIRASA
NLAATKMSECVLGQSKRVDFCGKGYHLMSFPQSAPHGVVF
LHVTYVPAQEKNFTTAPAICHDGKAHFPREGVFVSNGTHW
FVTQRNFYEPQIITTDNTFVSGNCDVVIGIVNNTVYDPLQ
PELDSFKEELDKYFKNHTSPDVDLGDISGINASVVNIQKE
IDRLNEVAKNLNESLIDLQELGKYEQ
USEO1_5P_DS3
linker underlined, cysteine bolded,
proline modifications
bold underlined
(SEQ ID NO: 15)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGGSSIIAYTMSLGAENSV
ACSNNSIAIPTNFTISVTTEILPVSMTKTSVDCTMYICGD
STECSNLLLQYGSFCTQLNRALTGIAVEQDKNTQEVFAQV
KQIYKTPPIKDFGGFNFSQILPDPSKPSKRSPIEDLLFNK
VTLADAGFIKQYGDCLGDIAARDLICAQKFNGLTVLPPLL
TDEMIAQYTSALLAGTICSGWTFGAGPALQIPFPMQMAYR
ENGIGVTQNVLYENQKLIANQFNSAIGKIQDSLSSTPSAL
GKLQDVVNQNAQALNTLVKQLSSNFGAISSVLNDILSRLD
KPEAEVQIDRLITGRLQSLQTYVTQQLIRAAEIRASANLA
ATKMSECVLGQSKRVDFCGKGYHLMSFPQSAPHGVVFLHV
TYVPAQEKNFTTAPAICHDGKAHFPREGVFVSNGTHWFVT
QRNFYEPQIITTDNTFVSGNCDVVIGIVNNTVYDPLQPEL
DSFKEELDKYFKNHTSPDVDLGDISGINASVVNIQKEIDR
LNEVAKNLNESLIDLQELGKYEQ
USEO4_5P_DS3
linker underlined, cysteine bolded,
proline modifications
bold underlined
(SEQ ID NO: 16)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
DVNCTEVPVAIHADQLTPTWRVYSTGSNVFQTRAGCLIGA
EHVNNSYECDIPIGAGICASYQGPGPSIIAYTMSLGAENS
VACSNNSIAIPTNFTISVTTEILPVSMTKTSVDCTMYICG
DSTECSNLLLQYGSFCTQLNRALTGIAVEQDKNTQEVFAQ
VKQIYKTPPIKDFGGFNFSQILPDPSKPSKRSPIEDLLFN
KVTLADAGFIKQYGDCLGDIAARDLICAQKFNGLTVLPPL
LTDEMIAQYTSALLAGTICSGWTFGAGPALQIPFPMQMAY
RFNGIGVTQNVLYENQKLIANQFNSAIGKIQDSLSSTPSA
LGKLQDVVNQNAQALNTLVKQLSSNFGAISSVLNDILSRL
DKPEAEVQIDRLITGRLQSLQTYVTQQLIRAAEIRASANL
AATKMSECVLGQSKRVDFCGKGYHLMSFPQSAPHGVVFLH
VTYVPAQEKNFTTAPAICHDGKAHFPREGVFVSNGTHWFV
TQRNFYEPQIITTDNTFVSGNCDVVIGIVNNTVYDPLQPE
LDSFKEELDKYFKNHTSPDVDLGDISGINASVVNIQKEID
RLNEVAKNLNESLIDLQELGKYEQ
PentaPro_D614G
proline modifications and
D614G mutation bold underlined
(SEQ ID NO: 17)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
GVNCTEVPVAIHADQLTPTWRVYSTGSNVFQTRAGCLIGA
EHVNNSYECDIPIGAGICASYQTQTNSPGSASSVASQSII
AYTMSLGAENSVAYSNNSIAIPTNFTISVTTEILPVSMTK
TSVDCTMYICGDSTECSNLLLQYGSFCTQLNRALTGIAVE
QDKNTQEVFAQVKQIYKTPPIKDFGGFNFSQILPDPSKPS
KRSPIEDLLFNKVTLADAGFIKQYGDCLGDIAARDLICAQ
KFNGLTVLPPLLTDEMIAQYTSALLAGTITSGWTFGAGPA
LQIPFPMQMAYRFNGIGVTQNVLYENQKLIANQFNSAIGK
IQDSLSSTPSALGKLQDVVNQNAQALNTLVKQLSSNFGAI
SSVLNDILSRLDKPEAEVQIDRLITGRLQSLQTYVTQQLI
RAAEIRASANLAATKMSECVLGQSKRVDFCGKGYHLMSFP
QSAPHGVVFLHVTYVPAQEKNFTTAPAICHDGKAHFPREG
VFVSNGTHWFVTQRNFYEPQIITTDNTFVSGNCDVVIGIV
NNTVYDPLQPELDSFKEELDKYFKNHTSPDVDLGDISGIN
ASVVNIQKEIDRLNEVAKNLNESLIDLQELGKYEQ
USEO1_5P_DS3_D614G
linker underlined, cysteine
bolded, proline
modifications and D614G
mutation bold underlined
(SEQ ID NO: 18)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
GVNCTEVPVAIHADQLTPTWRVYSTGSNVFQTRAGCLIGA
EHVNNSYECDIPIGAGICASYQGGSSIIAYTMSLGAENSV
ACSNNSIAIPTNFTISVTTEILPVSMTKTSVDCTMYICGD
STECSNLLLQYGSFCTQLNRALTGIAVEQDKNTQEVFAQV
KQIYKTPPIKDFGGFNFSQILPDPSKPSKRSPIEDLLFNK
VTLADAGFIKQYGDCLGDIAARDLICAQKFNGLTVLPPLL
TDEMIAQYTSALLAGTICSGWTFGAGPALQIPFPMQMAYR
ENGIGVTQNVLYENQKLIANQFNSAIGKIQDSLSSTPSAL
GKLQDVVNQNAQALNTLVKQLSSNFGAISSVLNDILSRLD
KPEAEVQIDRLITGRLQSLQTYVTQQLIRAAEIRASANLA
ATKMSECVLGQSKRVDFCGKGYHLMSFPQSAPHGVVFLHV
TYVPAQEKNFTTAPAICHDGKAHFPREGVFVSNGTHWFVT
QRNFYEPQIITTDNTFVSGNCDVVIGIVNNTVYDPLQPEL
DSFKEELDKYFKNHTSPDVDLGDISGINASVVNIQKEIDR
LNEVAKNLNESLIDLQELGKYEQ
USEO3_5P_DS3_D614G
linker underlined, cysteine bolded, proline
modifications bold underlined
(SEQ ID NO: 19)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVFNATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
GVNCTEVPVAIHADQLTPTWRVYSTGSNVFQTRAGCLIGA
EHVNNSYECDIPIGAGICASYQGPSIIAYTMSLGAENSVA
CSNNSIAIPTNFTISVTTEILPVSMTKTSVDCTMYICGDS
TECSNLLLQYGSFCTQLNRALTGIAVEQDKNTQEVFAQVK
QIYKTPPIKDFGGFNFSQILPDPSKPSKRSPIEDLLENKV
TLADAGFIKQYGDCLGDIAARDLICAQKFNGLTVLPPLLT
DEMIAQYTSALLAGTICSGWTFGAGPALQIPFPMQMAYRF
NGIGVTQNVLYENQKLIANQFNSAIGKIQDSLSSTPSALG
KLQDVVNQNAQALNTLVKQLSSNFGAISSVLNDILSRLDK
PEAEVQIDRLITGRLQSLQTYVTQQLIRAAEIRASANLAA
TKMSECVLGQSKRVDFCGKGYHLMSFPQSAPHGVVFLHVT
YVPAQEKNFTTAPAICHDGKAHFPREGVFVSNGTHWFVTQ
RNFYEPQIITTDNTFVSGNCDVVIGIVNNTVYDPLQPELD
SFKEELDKYFKNHTSPDVDLGDISGINASVVNIQKEIDRL
NEVAKNLNESLIDLQELGKYEQ
USEO4_5P_DS3_D614G
linker underlined, cysteine
bolded, proline
modifications and D614
mutation bold underlined
(SEQ ID NO: 20)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
GVNCTEVPVAIHADQLTPTWRVYSTGSNVFQTRAGCLIGA
EHVNNSYECDIPIGAGICASYQGPGPSIIAYTMSLGAENS
VACSNNSIAIPTNFTISVTTEILPVSMTKTSVDCTMYICG
DSTECSNLLLQYGSFCTQLNRALTGIAVEQDKNTQEVFAQ
VKQIYKTPPIKDFGGFNFSQILPDPSKPSKRSPIEDLLFN
KVTLADAGFIKQYGDCLGDIAARDLICAQKFNGLTVLPPL
LTDEMIAQYTSALLAGTICSGWTFGAGPALQIPFPMQMAY
RFNGIGVTQNVLYENQKLIANQFNSAIGKIQDSLSSTPSA
LGKLQDVVNQNAQALNTLVKQLSSNFGAISSVLNDILSRL
DKPEAEVQIDRLITGRLQSLQTYVTQQLIRAAEIRASANL
AATKMSECVLGQSKRVDFCGKGYHLMSFPQSAPHGVVFLH
VTYVPAQEKNFTTAPAICHDGKAHFPREGVFVSNGTHWFV
TQRNFYEPQIITTDNTFVSGNCDVVIGIVNNTVYDPLQPE
LDSFKEELDKYFKNHTSPDVDLGDISGINASVVNIQKEID
RLNEVAKNLNESLIDLQELGKYEQ
USEO5_5P_DS3_D614G
linker underlined, cysteine
bolded, proline
modifications and D614G
mutation bold underlined
(termed VFLIP_D614G)
(SEQ ID NO: 21)
QCVNLTTRTQLPPAYTNSFTRGVYYPDKVFRSSVLHSTQD
LFLPFFSNVTWFHAIHVSGTNGTKRFDNPVLPFNDGVYFA
STEKSNIIRGWIFGTTLDSKTQSLLIVNNATNVVIKVCEF
QFCNDPFLGVYYHKNNKSWMESEFRVYSSANNCTFEYVSQ
PFLMDLEGKQGNFKNLREFVFKNIDGYFKIYSKHTPINLV
RDLPQGFSALEPLVDLPIGINITRFQTLLALHRSYLTPGD
SSSGWTAGAAAYYVGYLQPRTFLLKYNENGTITDAVDCAL
DPLSETKCTLKSFTVEKGIYQTSNFRVQPTESIVRFPNIT
NLCPFGEVENATRFASVYAWNRKRISNCVADYSVLYNSAS
FSTFKCYGVSPTKLNDLCFTNVYADSFVIRGDEVRQIAPG
QTGKIADYNYKLPDDFTGCVIAWNSNNLDSKVGGNYNYLY
RLFRKSNLKPFERDISTEIYQAGSTPCNGVEGENCYFPLQ
SYGFQPTNGVGYQPYRVVVLSFELLHAPATVCGPKKSTNL
VKNKCVNFNFNGLTGTGVLTESNKKFLPFQQFGRDIADTT
DAVRDPQTLEILDITPCSFGGVSVITPGTNTSNQVAVLYQ
GVNCTEVPVAIHADQLTPTWRVYSTGSNVFQTRAGCLIGA
EHVNNSYECDIPIGAGICASYQGGSGGSSIIAYTMSLGAE
NSVACSNNSIAIPTNFTISVTTEILPVSMTKTSVDCTMYI
CGDSTECSNLLLQYGSFCTQLNRALTGIAVEQDKNTQEVF
AQVKQIYKTPPIKDFGGFNFSQILPDPSKPSKRSPIEDLL
ENKVTLADAGFIKQYGDCLGDIAARDLICAQKFNGLTVLP
PLLTDEMIAQYTSALLAGTICSGWTFGAGPALQIPFPMQM
AYRENGIGVTQNVLYENQKLIANQFNSAIGKIQDSLSSTP
SALGKLQDVVNQNAQALNTLVKQLSSNFGAISSVLNDILS
RLDKPEAEVQIDRLITGRLQSLQTYVTQQLIRAAEIRASA
NLAATKMSECVLGQSKRVDFCGKGYHLMSFPQSAPHGVVF
LHVTYVPAQEKNFTTAPAICHDGKAHFPREGVFVSNGTHW
FVTQRNFYEPQIITTDNTFVSGNCDVVIGIVNNTVYDPLQ
PELDSFKEELDKYFKNHTSPDVDLGDISGINASVVNIQKE
IDRLNEVAKNLNESLIDLQELGKYEQ
USEO5_5P_DS2
linker underlined, cysteine bolded
(SEQ ID NO: 22)
QCVNLTTRTQLPPAYTNSFTRGVYYPDKVFRSSVLHSTQD
LFLPFFSNVTWFHAIH
VSGTNGTKRFDNPVLPFNDGVYFASTEKSNIIRGWIFGTT
LDSKTQSLLIVNNATNVVIKVCEFQFCNDPFLGVYYHKNN
KSWMESEFRVYSSANNCTFEYVSQPFLMDLEGKQGNFKNL
REFVFKNIDGYFKIYSKHTPINLVRDLPQGFSALEPLVDL
PIGINITRFQTLLALHRSYLTPGDSSSGWTAGAAAYYVGY
LQPRTFLLKYNENGTITDAVDCALDPLSETKCTLKSFTVE
KGIYQTSNFRVQPTESIVRFPNITNLCPFGEVENATRFAS
VYAWNRKRISNCVADYSVLYNSASFSTFKCYGVSPTKLND
LCFTNVYADSFVIRGDEVRQIAPCQTGKIADYNYKLPDDF
TGCVIAWNSNNLDSKVGGNYNYLYRLFRKSNLKPFERDIS
TEIYQAGSTPCNGVEGENCYFPLQSYGFQPTNGVGYQPYR
VVVLSFELLHAPATVCGPKKSTNLVKNKCVNFNFNGLTGT
GVLTESNKKFLPFQQFGRDIADTTDAVRDPQTLEILDITP
CSFGGVSVITPGTNTSNQVAVLYQDVNCTEVPVAIHADQL
TPTWRVYSTGSNVFQTRAGCLIGAEHVNNSYECDIPIGAG
ICASYQGGSGGSSSIIAYTMSLGAENSVAYSNNSIAIPTN
FTISVTTEILPVSMTKTSVDCTMYICGDSTECSNLLLQYG
SFCTQLNRALTGIAVEQDKNTQEVFAQVKQIYKTPPIKDF
GGFNFSQILPDPSKPSKRSPIEDLLFNKVTLADAGFIKQY
GDCLGDIAARDLICAQKFNGLTVLPPLLTDEMIAQYTSAL
LAGTITSGWTFGAGPALQIPFPMQMAYRENGIGVTQNVLY
ENQKLIANQFNSAIGKIQDSLSSTPSALGKLQDVVNQNAQ
ALNTLVKQLSSNFGAISSVLNDILSRLDCPEAEVQIDRLI
TGRLQSLQTYVTQQLIRAAEIRASANLAATKMSECVLGQS
KRVDFCGKGYHLMSFPQSAPHGVVFLHVTYVPAQEKNFTT
APAICHDGKAHFPREGVFVSNGTHWFVTQRNFYEPQIITT
DNTFVSGNCDVVIGIVNNTVYDPLQPELDSFKEELDKYFK
NHTSPDVDLGDISGINASVVNIQKEIDRLNEVAKNLNESL
IDLQELGKYEQ

Bacterial strains. E. coli strain Rosetta DE3 (Novagen) was grown in lysogeny broth. The genotype is: F-ompT hsdSB(rB-mB-) gal dcm (DE3) pRARE (CamR). Selection markers were used at the indicated concentrations: ampicillin (100 μg/mL); chloramphenicol (28.3 μg/mL).

Cell lines. HEK-293T, Vero E6 and Vero-CCL81 cell lines were obtained from ATCC and cultured in DMEM medium (Gibco 31966021) supplemented with 10% Fetal Bovine Serum and incubated at 37° C. and 5% CO2. HEK-293F and Expi-CHO cells were obtained from Thermo Fisher Scientific and maintained in Expi293 Expression Medium and ExpiCHO-Expression Medium (Thermo Fisher Scientific), respectively. Design of SARS-CoV-2 spike variants. Spike variants were initially designed using the S-2P construct that includes ectodomain residues 13-1208 (Genbank: MN908947), two proline substitutions (K986P, V967P), and substitution of cleavage site residues RRAR with GSAS at position 682-685 (“RRAR” to “GSAS”). Designs containing five or six prolines were based on the HexaPro construct that carries, in addition to the proline substitutions of S-2P, proline substitutions at positions 817, 892, 899 and 942. All variants were cloned into a PhCMV mammalian expression vector containing a C-terminal foldon trimerization domain followed by an HRV-3C cleavage site and a Twin-Strep-Tag. Using the published S-2P cryo-EM structure (PDB: 6VSB), truncated cleavage site linkers of varying length and flexibility were designed to prevent S1/S2 cleavage and improve protein expression. Candidate interprotomer disulfide bond candidates were selected by assessing residues with CB atoms lying within 5 A of subunit interfaces, or by visual inspection. HexaPro P986 was reverted to lysine to restore a potential interprotomer salt-bridge that is disrupted by this mutation (PDB: 6VXX). Combinatorial variants containing different cleavage site linkers, proline substitutions and disulfide bonds were evaluated for effects on purity, yield and thermostability.

Transient transfection and protein purification. SARS-CoV-2 spike variants were transiently transfected in Freestyle 293-F and ExpiCHO-S cells (Thermo Fisher). Both cell lines were maintained and transfected according to the manufacturer's protocols. Briefly, 293-F cells were transfected with plasmid DNA mixed with polyethylenimine and harvested on day 5. Cultures were clarified by centrifugation, followed by addition of BioLock (IBA Life Sciences), passage through a 0.22 μM sterile filter, and purification on an ÄKTA go system (Cytivia) using a 5 mL StrepTrap-HP column equilibrated with TBS buffer (25 mM Tris pH 7.6, 200mM NaCl, 0.02% NaN3), and eluted in TBS buffer supplemented with 5 mM d-desthiobiotin (Sigma Aldrich). Proteins were then purified by size-exclusion-chromatography (SEC) on a Superdex 6 Increase 10/300 column (Cytivia) in the same TBS buffer.

For all ExpiCHO cultures, the manufacturer's “High Titer” protocol was used with a 7-day culture incubation to assess relative expression. Briefly, plasmid DNA and Expifectamine were mixed in Opti-PRO SFM (Gibco) according to the manufacturer's instructions, and added to the cells. On day 1, cells were fed with manufacturer-supplied feed and enhancer as specified in the manufacturer's protocol, and cultures were moved to a shaker incubator set to 32° C., 5% CO2 and 115 RPM. On day 7, the cultures were clarified by centrifugation, BioLock was added, and supernatants were passed through a 0.22 μM sterile filter. Purification was performed as above, on an ÄKTA go system using a 5 mL StrepTrap HP column and TBS buffer, followed by SEC on a Superdex 6 Increase 10/300 column with TB S buffer.

Differential Scanning calorimetry. Thermal stability was measured using a MicroCal VP-Capillary calorimeter (Malvern) with 0.6 mg/ml of each sample in phosphate-buffered saline (PBS) buffer at a scanning rate of 90° C. hour-1 from 20° C. to 120° C. Data were analyzed using VP-Capillary DSC automated data analysis software.

Negative stain electron microscopy (NS-EM). To prepare samples for NS-EM, 3 μL 0.02 mg/mL protein was applied to a carbon film 400 mesh grid for 1 minute. The grid was then washed three times with 10 μL Milli-Q water and stained three times with 4 μL drops of 1% uranyl formate, with the first two drops briefly and the third time for 1 minute. The grid was blotted with Whatman filter paper after each application of liquid. Micrographs were collected on a Titan Halo transmission electron microscope, operating with an accelerating voltage of 300 kV and using a pixel size of 1.4 Å/pixel.

Cryo-Electron Microscopy sample preparation. VFLIP and VFLIP_D614G were concentrated to 2 mg/ml and electron microscopy grids were prepared by placing a 3 μL aliquot of the sample on a plasma-cleaned C-flat grid (2/1C-3T, Protochips Inc) that was then immersed in liquid ethane for vitrification. For formation of VFLIP_D614G:HLX70 Fab and VFLIP_D614G:HLX71_ACE2-Fc complexes, the trimerization tag ‘Foldon’ and purification tags of VFLIP_D614G spike were enzymatically removed by an overnight treatment with HRV protease at room temperature before concentration to 1 mg/ml and incubation at a 1:2 molar ratio with HLX70 Fab or HLX71 ACE2-Fc at room temperature overnight. The samples were then injected over a gel filtration column (Superose 6 10/30, GE Life Sciences) equilibrated with 20 mM Tris pH 8.0 and 150 mM NaCl. The complex peak fractions were concentrated to an absorbance of 2.0. Electron microscopy grids were prepared as described above.

Cryo-EM data collection and processing. Grids were loaded into a Titan Krios G3 electron microscope (Thermo Fisher Scientific) equipped with a K3 direct electron detector (Gatan, Inc.) at the end of a BioQuantum energy filter, using an energy slit of 20 eV. The microscope was operated with an accelerating voltage of 300 kV. Grids were imaged with a pixel size of 0.66 Å in counting mode. Data was acquired using the software EPU. Motion correction, CTF estimation, and particle-picking were done with Warp (Tegunov and Cramer 2019). Extracted particles were exported to cryoSPARC-v2 (Punjani et al. 2017) (Structura Biotechnology Inc.) for 2D classification, ab initio 3D reconstruction, and refinement. Cl symmetry was used during homogeneous refinement.

Cryo-EM model building and analysis. A previously published structure of the SARS-CoV-2 ectodomain with all RBDs in the down conformation (PDB ID 6X79) was used to fit the cryo-EM maps in UCSF ChimeraX (Goddard et al. 2018) and PyMOL. Mutations were made in PyMOL. Coordinates were then fitted manually using COOT (Emsley et al. 2010), followed by cycles of refinement using Phenix (Afonine et al. 2018) real space refinement. COOT was used for subsequent fitting.

Glycoproteomics sample preparation. Recombinant SARS-CoV-2 spike protein was denatured at 95° C. at a final concentration of 2% sodium deoxycholate (SDC), 200 mM Tris/HCl, 10 mM tris(2-carboxyethyl)phosphine, pH 8.0 for 10 min, followed by a 30 min reduction at 37° C. Next, samples were alkylated by adding 40 mM iodoacetamide and incubated in the dark at room temperature for 45 min. For each protease digestion, 3 μg recombinant SARS-CoV-2 spike protein was used. Samples were divided in thirds for parallel digestion with gluC (Sigma)-trypsin (Promega), chymotrypsin (Sigma) and alpha lytic protease (Sigma). For each protease digestion, 18 μL of the denatured, reduced, and alkylated samples was diluted in a total volume of 100 μL 50 mM ammonium bicarbonate and proteases were added at a 1:30 ratio (w:w) for incubation overnight at 37° C. For the gluC-trypsin digestion, gluC was added first for two hours, and then incubated with trypsin overnight. After overnight digestion, SDC was removed by precipitation with 2 μL formic acid and centrifugation at 14,000 rpm for 20 min. The resulting supernatant containing the peptides was collected for desalting on a 30 μm Oasis HLB 96-well plate (Waters). The Oasis HLB sorbent was activated with 100% acetonitrile and subsequently equilibrated with 10% formic acid in water. Next, peptides were bound to the sorbent, washed twice with 10% formic acid in water and eluted with 100 μL 50% acetonitrile/10% formic acid in water (v/v). The eluted peptides were dried under vacuum and resuspended in 100 μL 2% formic acid in water. The experiment was performed in duplicate.

Glycoproteomics mass spectrometry. The duplicate samples were analyzed with two different mass spectrometry methods, using identical LC-MS parameters and distinct fragmentation schemes. In one method, peptides were subjected to Electron Transfer/Higher-Energy Collision Dissociation fragmentation (Frese et al. 2012, 2013). In the other method, all precursors were subjected to HCD fragmentation, with additional EThcD fragmentation triggered by the presence of glycan reporter oxonium ions. For each duplicate sample injection, approximately 0.15 μg of peptides were run on an Orbitrap Fusion Tribrid mass spectrometer (Thermo Fisher Scientific, Bremen) coupled to a Dionex UltiMate 3000 (Thermo Fisher Scientific). A 90-min LC gradient from 0% to 44% acetonitrile was used to separate peptides at a flow rate of 300 nl/min. Peptides were separated using a Poroshell 120 EC-C18 2.7-Micron analytical column (ZORBAX Chromatographic Packing, Agilent) and a C18 PepMap 100 trap column (5 mm×300 μm, 5 μm, Thermo Fisher Scientific). Data was acquired in data-dependent mode. Orbitrap Fusion parameters for the full scan MS spectra were as follows: a standard AGC target at 60 000 resolution, scan range 350-2000 m/z, Orbitrap maximum injection time 50 ms. The ten most intense ions (2+ to 8+ ions) were subjected to fragmentation. For the EThcD fragmentation scheme, the supplemental higher energy collision dissociation energy was set at 27%. MS2 spectra were acquired at a resolution of 30,000 with an AGC target of 800%, maximum injection time 250 ms, scan range 120-4000 m/z and dynamic exclusion of 16 s. For the triggered HCD-EThcD method, the LC gradient and MS1 scan parameters were identical. The ten most intense ions (2+ to 8+) were subjected to HCD fragmentation with 30% normalized collision energy from 120-4000 m/z at 30,000 resolution with an AGC target of 100% and a dynamic exclusion window of 16 s. Scans containing any of the following oxonium ions within 20 ppm were followed up with additional EThcD fragmentation with 27% supplemental HCD fragmentation. The triggering reporter ions were: Hex(1) (129.039; 145.0495; 163.0601), PHex(1) (243.0264; 405.0793), HexNAc(1) (138.055; 168.0655; 186.0761), Neu5Ac(1) (274.0921; 292.1027), Hex(1)HexNAc(1) (366.1395), HexNAc(2) (407.166), dHex(1)Hex(1)HexNAc(1) (512.1974), and Hex(1)HexNAc(1)Neu5Ac(1) (657.2349). EThcD spectra were acquired at a resolution of 30,000 with a normalized AGC target of 400%, maximum injection time 250 ms, and scan range 120-4000 m/z.

Mass spectrometry data analysis. The acquired data was analyzed using Byonic (v3.11.1) against a custom database of SARS-CoV-2 spike protein sequences and the proteases used in the experiment to search for glycan modifications with 12/24 ppm search windows for MS1 and MS2, respectively. Up to five missed cleavages were permitted using C-terminal cleavage at R/K/E/D for gluC-trypsin or F/Y/W/M/L for chymotrypsin. Up to 8 missed cleavages were permitted using C-terminal cleavage at T/A/S/V for alpha lytic protease. Carbamidomethylation of cysteine was set as a fixed modification and oxidation of methionine/tryptophan was set as variable rare 1. N-glycan modifications were set as variable common 2, allowing up to a maximum of 3 variable common and 1 rare modification per peptide. All N-linked glycan databases from Byonic were merged into a single non-redundant list for inclusion in the database search. All reported glycopeptides in the Byonic result files were first filtered for score ≥100 and PEP2D ≤0.01, then manually inspected for quality of fragment assignments. All glycopeptide identifications were merged into a single non-redundant list per sequon. Glycans were classified based on HexNAc and Hexose content as paucimannose (2 HexNAc, 3 Hex), high-mannose (2 HexNAc; >3 Hex), hybrid (3 HexNAc) or complex (>3 HexNAc). Byonic search results were exported into mzIdentML format to build a spectral library in Skyline (v20.1.0.31) and to extract peak areas for individual glycoforms from MS1 scans. N-linked glycan modifications identified from Byonic were manually added to the Skyline project file in XML format. Reported peak areas were pooled based on the number of HexNAc, Fuc or NeuAc residues to distinguish paucimannose, high-mannose, hybrid, and complex glycosylation.

High-throughput surface plasmon resonance. High-throughput SPR capture kinetic experiments were performed on an LSA biosensor system equipped with a planar carboxymethyldextran CMDP sensor chip (Carterra). The LSA automates the choreography between two microfluidic modules, namely a single flow cell (SFC), which flows samples over the entire array surface and a 96-channel printhead (96 PH) used to create arrays of up 384 samples. The capture surface was prepared using the SFC by standard amine-coupling of goat anti-human IgG Fc (Southern Biotech) to create a uniform surface, or lawn, over the entire chip. The system running buffer was 1× HBSTE (10 mM HEPES pH 7.4, 150 mM NaCl, 3 mM EDTA, 0.05% Tween-20). The chip was activated with a 10-minute

injection of freshly prepared 1:1:1 (v/v/v) 0.4M EDC 0.1 MN-hydroxysulfosuccinimide (SNHS) with 0.1M 2-(N-morpholino)ethanesulfonic acid (MES) pH 5.5 before coupling of goat anti-human IgG Fc (50 μg/ml in 10 mM sodium acetate pH 4.5) for 15 minutes. Excess reactive esters were blocked with a 7-minute injection of 1M ethanolamine HCl pH 8.5. Final coupled levels (mean±Std.Dev. RU across all 384 array regions of interest (ROIs) were 535±32 RU. After preparing the capture surface, the instrument was primed using assay running buffer (HBSTE with 0.5 mg/mL BSA). The Fc-ligands and mAbs were diluted into assay running buffer and captured onto the array using the 96PH for 15 minutes at three dilutions of 25, 3.6, and 0.9. Antibodies were captured and buffer blanks were then injected followed by a titration series of increasing antigen concentration. RBD and spike proteins were injected at 0.8, 2.5, 7.4, 22, 67, and 200 nM for 5 minutes with a 15-minute dissociation. After each antigen titration series the surface was regenerated with three, 60-second pulses of 0.475% H3PO4. Binding data from the local reference spots (interspots, representing the unoccupied capture surface) were subtracted from the active ROIs and the nearest buffer blank analyte responses were subtracted to double-reference the data. The double-referenced data were fit globally to a simple 1:1 Langmuir binding model using the Carterra Kinetics software tool to provide ka, kd, and Rmax values for each spot.

Biophysical experiments and spike-splitting tests of VFLIPΔFoldon. Enzymatic removal of the ‘Foldon’ trimerization tag from VFLIP and HexaPro was facilitated by cloning the HRV-C3 cleavage site followed by Strep purification tags between the C terminus of the SARS-CoV-2 spike and the Foldon. After purification on a StrepTrap HP column, proteins were incubated overnight at room temperature with 2U HRV-3C protease per 100 μg protein at room temperature. The cleaved proteins were then SEC purified. For biophysical characterization, 150 μg of each protein was incubated at 4° C. and 37° C. for 5 days and then SEC purified. The same amount of protein was subjected to 10 cycles of fast freeze/thaw and then SEC purified. For lyophilization, 150 μg Foldon-free spike was dehydrated overnight using a SpeedVac RT and the lyophilized proteins were resuspended 5 days later in TBS before SEC purification.

To form immune complexes between shACE2 and B6 Fab fragments, and offer the greatest chance for spike separation was observed for these molecules with other forms of spike, we incubated complexes for 2 days at 4° C. at a 1:2 molar ratio with Foldon-free proteins concentrated to 1 mg/ml. Samples were purified by SEC as described above.

Mice immunization. For mouse immunization and serum extraction Institutional Animal Care and Use Committee (IACUC) guidelines were followed with animal subjects tested in the immunogenicity study. Six-week-old BALB/c mice were purchased from the Jackson Laboratory. The mice were housed in ventilated cages in environmentally controlled rooms at the LJI animal facility, in compliance with an approved IACUC protocol and AAALAC (Association for Assessment and Accreditation of Laboratory Animal Care) International guidelines. At week 0, each mouse was immunized with 25 μg of the indicated antigen in 50 μl PBS together 25 μl of the Magic Mouse CpG adjuvant (Creative technologies) and 25 μl aluminum hydroxide (Invivogen) administered by an intramuscular (i.m.) route. At week 4, the animals were boosted with the same antigen/adjuvant composition as used for the prime. At week 6, the animals were bled through the retro-orbital membrane using fractionator tubes. Sera were heat inactivated at 56° C. for 1 hour and stored at −80° C. until analysis.

Enzyme-linked immunosorbent assays. 96-well EIA/RIA plates (Corning, Sigma) were coated with 0.1 μg per well of HexaPro in PBS and incubated at 4° C. overnight. On the following day, the coating solution was removed and wells were blocked with 5% skim milk diluted in PBS with 0.1% Tween 20 (PBST) at room temperature for 1 h. Mouse serum samples that had been previously heat inactivated at 56° C. for 1 h were diluted 1:50 and the serially diluted five-fold in 5% skim milk in PBST. The blocking solution was removed and 50 μl of the diluted sera was added to the plates and incubated for 1 h at room temperature. Following incubation, the diluted sera were removed and the plates were washed 4 times with PBST. Goat anti-human IgG secondary antibody-peroxidase (Fc-specific, Sigma) diluted 1:3,000 in 5% skim milk in PB ST was then added and the plates were incubated for 1 h at room temperature before washing four times with PB ST. The ELISA was developed using 3,5,3′,5′-tetramethylbenzidine (Thermo Fisher Scientific) solution and the reaction was stopped after 5 min incubation with 4N sulfuric acid. The OD450 was measured using a

Tecan Spark 10M plate reader. The dilution of each serum sample required to obtain a 50% maximum signal (EC50) against HexaPro was determined using nonlinear regression analysis in Prism 8 version 8.4.2 (GraphPad).

rVSV SARS2 pseudovirus neutralization assay. Recombinant SARS-CoV-2-pseudotyped VSV-ΔG-GFP was generated by transfecting 293T cells with phCMV3-SARS-CoV-2 full-length spike carrying the D614G mutation and deletion of the 19 C-terminal amino acid using TransIT according to the manufacturer's instructions. At 24 hr post-transfection, cells were washed 2× with OptiMEM and then infected with rVSV-G pseudotyped ΔG-GFP parent virus (VSV-G*ΔG-GFP) at MOI=2 for 2 hours with rocking. The virus was then removed, and the cells were washed twice with OPTI-MEM containing 2% FBS (OPTI-2) before addition of fresh OPTI-2. Supernatants containing rVSV-SARS-2 pseudoviruses were removed 24 hours post-infection and clarified by centrifugation, pooled and stored at −80° C. until use.

SARS-CoV-2-pseudotyped VSV-AG-GFP was next titered in Vero cells (ATCC CCL-81). Cells were seeded in 96-well plates at a sufficient density to form a monolayer at the time of infection. 10-fold serial dilutions of pseudovirus were made and added to cells in triplicate wells. Infection was allowed to proceed for 16-18 hr at 37° C. before fixation of the cells with 4% PFA and staining with Hoechst (10 μg/mL) in PBS. Fixative/stain was replaced with PBS and pseudovirus titers were quantified as the number of GFP-positive cells (fluorescent forming units, ffu/mL) using a Celllnsight CX5 imager (Thermo Scientific) and automated enumeration of cells expressing GFP.

Mouse sera neutralization assays were performed with pre-titrated amounts of rVSV-SARS-CoV-2 pseudovirus with sera samples diluted 1:100 and serial four-fold dilutions. The pseudovirus and sera samples were incubated together at 37° C. for 1 hr before addition to confluent Vero monolayers in 96-well plates. The plates were incubated for 16-18 hrs at 37° C. in 5% CO2, and then the cells were fixed with4% paraformaldehyde and stained with 10 μg/mL Hoechst. Cells were imaged using a Celllnsight CX5 imager and infection was quantified by automated enumeration of total cells and those expressing GFP. Infection was normalized to the average number of cells infected with rVSV-SARS-CoV-2 incubated without sera, and pooled sera from untreated mice was used as control. Data are presented as neutralization IC50 titers calculated using “One-Site Fit LogIC50” regression in GraphPad Prism 9.0.

Plaque reduction neutralization (PRNT) assay. SARS-CoV-2 variant D614G was obtained through BEI Resources, NIAID, NIH: SARS-Related Coronavirus 2, Isolate Germany/BayPat1/2020, NR-52370. SARS-CoV-2 strain B1.351 was obtained through BEI Resources, NIAID, NIH: SARS-Related Coronavirus 2, Isolate hCoV-19/South Africa/KRISP-K005325/2020, NR-54009. Both SARS-CoV-2 D614G and B1.351 were propagated in Vero-CCL81 cells, titrated by plaque assay on Vero E6 cells, deep-sequenced by the La Jolla Institute for Immunology Sequencing Core. Assays were performed in the BSL3 facility at La Jolla Institute for Immunology. For PRNT assay, mouse serum was serial 5-fold diluted, starting from 50-fold to 156250-fold, before co-culture with 30-40 plaque forming units (PFU) of SARS-CoV-2 D614G or B1.351 for 1 h at 37° C. The serum/virus mixture was then transferred onto Vero E6 cells (8×104 cells/well, 24-well plate) for 2 h at 37° C. The inoculum was removed before overlaid with 1% carboxymethylcellulose medium to each well. All the conditions were tested in duplication. After 3 days cultivation, cells were fixed with 10% formaldehyde in PBS for 30 min at RT prior stained with 0.1% crystal violet solution for 20 min at RT. Serum titer (NT50) was determined as the highest sample dilution that neutralize 50% of virus plaques.

Multiple coronaviruses (CoVs) have emerged to cause outbreaks of human disease in the last 20 years. Among the genus Betacoronavirus, severe acute respiratory syndrome coronavirus (SARS-CoV, or SARS-CoV-1) emerged in China in 2002, spreading to 8,000 cases with 10% mortality before it was contained in 2003. A decade later, Middle East respiratory syndrome (MERS) coronavirus emerged in Saudi Arabia and ultimately infected 2,500 people across Europe, Asia, and the Middle East with 35% lethality. MERS is still recurrently introduced into human populations, with a recent report finding seropositivity among abattoir workers in Nigeria. Four other CoVs are endemic in the human population including the betacoronaviruses hCoV-OC43 and hCoV-HKU1. In late 2019, SARS-CoV-2 emerged in China and rapidly spread globally to infect nearly 120 million people and cause over 2 million deaths. Given the myriad novel CoVs identified in ecological sampling, yet another novel CoV will likely emerge in human populations.

In addition to CoVs that have yet to emerge, it is important to prepare broad neutralizing antibodies that can prevent not only the infectivity of existing CoVs that have already achieved sustained human-to-human transmission, but also future CoV variants. Among human SARS-CoV-2 infections and multiple instances of human/animal spillover and spillback, a series of amino acid substitutions, deletions and potential recombinations have created additional sequence diversity. The number of existing infections, ongoing asymptomatic spread, temporal and logistical challenges of widespread vaccine delivery and uptake, together with animal transmission events suggests that SARS-CoV-2 may become endemic and exhibit seasonal returns over the long term. Such seasonal return could be accompanied by additional mutagenic drift, necessitating either seasonal vaccination or development of vaccines that elicit broad and durable immunity. These concerns highlight the need to develop new and more powerful tools to study coronaviruses and to develop a novel set of antigens and prophylactic antibodies that can be rapidly advanced to clinical trials.

The spike (S) glycoprotein displayed on the virus surface in the “pre-fusion” metastable state, is the main target of neutralizing and protective antibodies for all the betacoronaviruses and therefore is also the main target for most CoV vaccine efforts. The spike is a trimer comprising heterodimers of S1 and S2 subunits that mediate receptor binding and membrane fusion, respectively. Upon receptor binding, the trimer of S1-S2 springs from a “pre-fusion” to “post-fusion” assembly that allows membrane fusion and subsequent viral entry. The pre-fusion spike assembly is inherently unstable, and when expressed outside the virion, or when whole virions are irradiated for vaccines, the spike can rapidly separate and spring into the post-fusion conformation. Thus, stabilized spike protein that maintains the pre-fusion conformation is essential for vaccine strategies to ensure that appropriate three-dimensional surfaces are displayed and can elicit protective antibodies. Most SARS-CoV-2 vaccine candidates now in use or in clinical trials employ an “S-2P” construct that: (i) is truncated at residue 1208; (ii) lacks the furin cleavage site at the S1/S2 boundary; and (iii) contains two proline substitutions (2P) at positions 986 and 987. Several S-2P based vaccine candidates showed initial promise, but the rapid development of these candidates afforded few opportunities for optimization.

Findings by Hsieh et al. indicate that S-2P spikes are characterized by poor yield, purity and thermal instability, which may all compromise manufacturability and deliverability, increase costs, and limit expression levels both in vaccinees and in cell culture. Amanat et al. showed that stabilization of spike is critical to induce protective immune responses. Other studies confirmed the temperature sensitivity of S-2P spike preparations as evidenced by a 95% loss of well-formed spike trimers after only 5-7 days storage at 4° C. The need for “cold-chain stability” is a major obstacle for mobilization of multiple types of vaccines. A second-generation HexaPro version carrying four additional prolines offers greater yield and stability relative to S-2P, but lacks a key salt bridge that mediates the pre-fusion quaternary structure. Moreover, HexaPro has glycan structures that do not reflect those of the authentic virus. Together, these sequence alterations and glycan structures may result in an immunogen that does not display surfaces targeted by protective antibodies. Thus, improved immunogens are needed. These results emphasize the need of a more quaternary structured for SARS-CoV-2 prefusion-stabilized S glycoprotein that retains the trimeric state and faithfully displays quaternary epitopes targeted by antibodies for use as a robust antigenic tool for structural characterization of a large population of neutralizing antibodies. These antibodies seem to cluster in 3 communities which, upon interaction with HexaPro spike, trigger fusogenic rearrangement of the protein that can complicate determination of high resolution structures of antibody-spike complexes.

To achieve a third-generation vaccine and produce a tool for structural analyses, the inventors undertook successful engineering of a thermostable glycoprotein spike involving a different pattern of proline mutations, adjusted subunit linkages and introduction of a disulfide bond in the inner core to preserve the quaternary assembly of the pre-fusion state. This novel construct is termed VFLIP, for (V-five) Flexibly-Linked, Inter-Protomer spike (SEQ ID NO:14). The inventors evaluated the biophysical properties of VFLIP and its recognition by neutralizing antibodies against SARS-CoV-2.

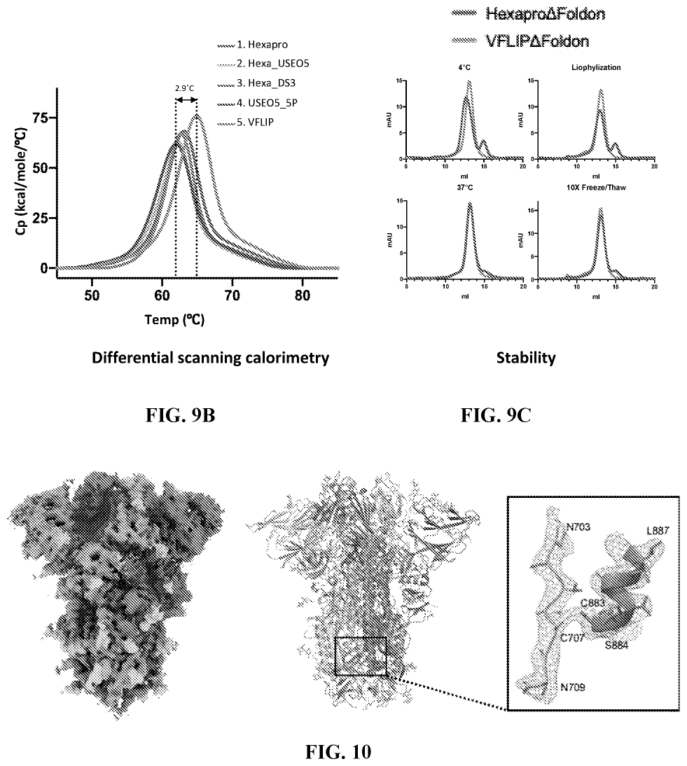

To create the SARS-CoV-2 VFLIP spike, the inventors made the following modifications: (i) reverted one of the six prolines of HexaPro (K986P) that could affect the quaternary structure by ablating the K986-E748 salt bridge within each protomer; (ii) introduced an inter-protomer disulfide bond between the S2 of one protomer (position 707) and the S2′ of the adjacent protomer (position 883); and (iii) replaced the S1/S2 cleavage fusion loop with a short flexible linker that demonstrated an additional stability and increased expression levels. The resulting VFLIP spike expresses to levels ten-fold higher than those for S-2P. VFLIP also offers a ˜3° C. improvement in thermostability (FIG. 9B) over HexaPro and, surprisingly, retains its trimeric structure even after lyophilization, freeze/thaw cycles, and prolonged storage at room temperature, 4° C. and 37° C., conditions that cause other versions of spike to separate into monomers (FIG. 9C). Notably, the introduction of the disulfide into the spike core promotes maintenance of the trimer structure without the need for an exogenous trimerization domain such as Foldon or the HIV-1-derived “molecular clamp” that triggered removal of the Australian SARS-CoV-2 vaccine candidate from clinical trials after the trimerization domain caused vaccine recipients to register false positives in HIV tests (FIGS. 9A to 9C).

Another benefit of VFLIP spike is that its glycan structures are more similar to those of spikes on the native virion. For example, in both VFLIP and authentic virus, complex glycans are present at N603 and N709 in the conserved S2 subunit, whereas HexaPro and S-2P spike instead display high-mannose structures.

This third-generation design of VFLIP spike allows presentation of the spike receptor-binding domains (RBDs) in conformations reflective of spike on the viral surface, i.e., all “down”, until interaction with the angiotensin-converting enzyme 2 (ACE2) receptor lifts the RBDs to the “up” conformation. In contrast, for second-generation HexaPro-like spikes, typically one or more RBDs is up, whereas other covalently-linked spikes have all RBDs “locked down” and often positioned deeper than on the native virion, which both can potentially affect the antigenicity of these designs. VFLIP also possesses a well-formed disulfide bond connecting two adjacent protomers that may result in more faithful display of quaternary epitopes (FIG. 10).

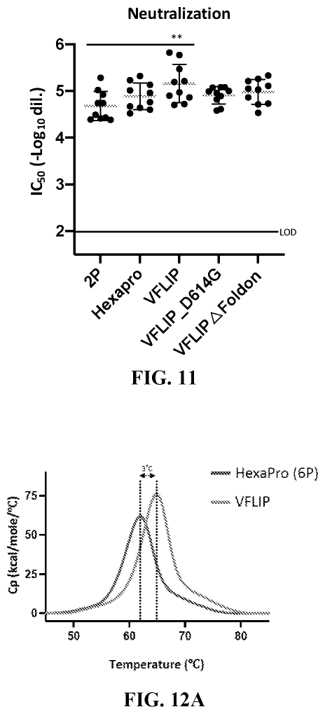

Finally, to assess how the VFLIP design may impact immunogenicity, the inventors immunized BALB/c mice with five constructs: (1) Parental S-2P, (2) HexaPro, (3) VFLIP, (4) VFLIP.D614G and (5) VFLIPΔFoldon adjuvanted with CpG+alum and boosted with the same four weeks later (FIG. 11). An interim blood draw was taken two weeks after the prime, and a final blood draw was taken two weeks after the boost. By way of explanation, and in no way a limitation of the present disclosure, the inventors hypothesized that the additional stability afforded by VFLIP may improve elicitation of neutralizing antibodies, and further hypothesized that removing the Foldon (VFLIPΔFoldon) may avoid deleterious responses to this exogenous trimerization domain. Al other constructs analyzed here (2P, Hexapro, etc. contain the trimerization domain. Only VFLIPΔFoldon has the trimerization domain removed.). Overall, all groups mounted appreciable antibody responses, but groups 2-5 all outperformed group 1 (S-2P) with significantly higher total anti-spike titers. Neutralization assays using rVSV-pseudotyped with SARS-CoV-2 spike bearing the G614 mutation were performed to measure the potency of the sera from immunized mice. Sera from all immunized groups displayed high IC50 values, with VFLIP groups showing significantly higher neutralizing titers than the 2P group.

Together, these results confirm that VFLIP is an excellent vaccine candidate for further study due to its exceptional stability, high yields and improved immunogenicity. The VFLIP technology can also be applied to other SARS-CoV-2 variants (including the B.1.1.7 (UK) and B.1.135 (South African) variants as well as to other human coronaviruses, including but not limited to B.1.1.7 with E484K, as well as B.1.617 (including both Delta an Kappa variants), B.1.351, P.1, B.1.427, B.1.429, Lambda (i.e. C.37), Mu (i.e. B.1.621), or other emerging variants of SARS-CoV-2. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), and Delta (B.1.617.2 and AY lineages). Thus, the VFLIP of the present disclosure can be used with any current or emerging variants. The skilled artisan will understand that the VFLIP technology is applicable to all variants, current or future, and a person skilled in the art, after learning of the VFLIP modifications thought herein, would be able to stabilize and improve expression of any yet-to-emerge Spike variants without undue experimentation.

Suitability of SARS-CoV-2 spike technology “VFLIP” for other key human coronaviruses.

The design strategies successfully used for VFLIP SARS-CoV-2 can be used to generate similar modified spikes for other relevant human CoVs. In developing VFLIP, the inventors evaluated the disulfide introduction in ten different spike variants before selecting Y707 and T883 located between the S2 and S2′ subunits in SARS-CoV-2 spike as the ideal combination to maximize yield, trimer propensity, thermal stability and native glycan incorporation. The inventors can introduce the VFLIP disulfide at equivalent positions in other CoV spikes: SARS-CoV (Y689-T865C), MERS-CoV (L780C-A968C), HKU1 (V779C-P970C), OC43 (L791C-P982C), in addition to the redesign of the corresponding cleavage site loop, and proline substitutions. Detailed sequences of the abovementioned SARS-CoV, MERS-CoV, HKU1 and OC43 spikes stabilized on the pre-fusion conformation with the VFLIP technology are as follows.

VFLIP technology applied to other coronaviruses and variants.

SARS2_VFLIP_South_African_variant (501Y.V2)

SARS-CoV-2 “spike” with the VFLIP technology, and the mutations
contained in the South African variant
(SEQ ID NO: 23)
QCVNFTTRTQLPPAYTNSFTRGVYYPDKVFRSSVLHSTQDLFLPFFSNVTWFHAIHVSGTN
GTKRFANPVLPFNDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNFKNLREFVFK
NIDGYFKIYSKHTPINLVRGLPQGFSALEPLVDLPIGINITRFQT...LHRSYLTPGDSSSGWTA
GAAAYYVGYLQPRTFLLKYNENGTITDAVDCALDPLSETKCTLKSFTVEKGIYQTSNFRV
QPTESIVRFPNITNLCPFGEVENATRFASVYAWNRKRISNCVADYSVLYNSASFSTFKCYG
VSPTKLNDLCFTNVYADSFVIRGDEVRQIAPGQTGNIADYNYKLPDDFTGCVIAWNSNNL
DSKVGGNYNYLYRLFRKSNLKPFERDISTEIYQAGSTPCNGVKGFNCYFPLQSYGFQPTYG
VGYQPYRVVVLSFELLHAPATVCGPKKSTNLVKNKCVNFNFNGLTGTGVLTESNKKFLPF
QQFGRDIADTTDAVRDPQTLEILDITPCSFGGVSVITPGTNTSNQVAVLYQGVNCTEVPVAI
HADQLTPTWRVYSTGSNVFQTRAGCLIGAEHVNNSYECDIPIGAGICASYQGGSGGSSIIA
YTMSLGVENSVACSNNSIAIPTNFTISVTTEILPVSMTKTSVDCTMYICGDSTECSNLLLQY
GSFCTQLNRALTGIAVEQDKNTQEVFAQVKQIYKTPPIKDFGGFNFSQILPDPSKPSKRSPIE
DLLFNKVTLADAGFIKQYGDCLGDIAARDLICAQKFNGLTVLPPLLTDEMIAQYTSALLAG
TICSGWTFGAGPALQIPFPMQMAYRFNGIGVTQNVLYENQKLIANQFNSAIGKIQDSLSSTP
SALGKLQDVVNQNAQALNTLVKQLSSNFGAISSVLNDILSRLDKPEAEVQIDRLITGRLQS
LQTYVTQQLIRAAEIRASANLAATKMSECVLGQSKRVDFCGKGYHLMSFPQSAPHGVVFL
HVTYVPAQEKNFTTAPAICHDGKAHFPREGVFVSNGTHWFVTQRNFYEPQIITTDNTFVSG
NCDVVIGIVNNTVYDPLQPELDSFKEELDKYFKNHTSPDVDLGDISGINASVVNIQKEIDRL
NEVAKNLNESLIDLQELGKYEQYIKGSGYIPEAPRDGQAYVRKDGEWVLLSTELLEVLFQGPA
GWSHPQFEKGGGSGGGSGGGSWSHPQFEK

Underlined are the 8 mutations contained in SARS-CoV-2 501Y.V2 variant is a deletion of three amino acids over the Wuhan variant. Italics are amino acids are exogenous tags for stabilization and purification purposes

SARS1_VFLIP
(SEQ ID NO: 24)
SDLDRCTTFDDVQAPNYTQHTSSMRGVYYPDEIFRSDTLYLTQDLFLPFYSNVTGFHTINHT
FDNPVIPFKDGIYFAATEKSNVVRGWVFGSTMNNKSQSVIIINNSTNVVIRACNFELCDNPFF
AVSKPMGTQTHTMIFDNAFNCTFEYISDAFSLDVSEKSGNFKHLREFVFKNKDGFLYVYKG
YQPIDVVRDLPSGFNTLKPIFKLPLGINITNFRAILTAFSPAQDTWGTSAAAYFVGYLKPTTF
MLKYDENGTITDAVDCSQNPLAELKCSVKSFEIDKGIYQTSNFRVVPSGDVVRFPNITNLCP
FGEVFNATKFPSVYAWERKKISNCVADYSVLYNSTFFSTFKCYGVSATKLNDLCFSNVYAD
SFVVKGDDVRQIAPGQTGVIADYNYKLPDDFMGCVLAWNTRNIDATSTGNYNYKYRYLR
HGKLRPFERDISNVPFSPDGKPCTPPALNCYWPLNDYGFYTTTGIGYQPYRVVVLSFELLNA
PATVCGPKLSTDLIKNQCVNFNFNGLTGTGVLTPSSKRFQPFQQFGRDVSDFTDSVRDPKTS
EILDISPCSFGGVSVITPGTNASSEVAVLYQDVNCTDVSTAIHADQLTPAWRIYSTGNNVFQ
TQAGCLIGAEHVDTSYECDIPIGAGICASYHGGSGGSSIVAYTMSLGADSSIACSNNTIAIPT
NFSISITTEVMPVSMAKTSVDCNMYICGDSTECANLLLQYGSFCTQLNRALSGIAAEQDRNT
REVFAQVKQMYKTPTLKYFGGFNFSQILPDPLKPTKRSPIEDLLFNKVTLADAGFMKQYGE
CLGDINARDLICAQKFNGLTVLPPLLTDDMIAAYTAALVSGTACAGWTFGAGPALQIPFPM
QMAYRFNGIGVTQNVLYENQKQIANQFNKAISQIQESLTTTPTALGKLQDVVNQNAQALN
TLVKQLSSNFGAISSVLNDILSRLDKPEAEVQIDRLIYIKGSGYIPEAPRDGQAYVRKDGEWVLL
STFLLEVLFQGPAGWSHPQFEKGGGSGGGSGGGSWSHPQFEK

Bold are the stabilizing mutations (VLIP technology) applied to SARS-CoV-1

Italics are amino acids are exogenous tags for stabilization and purification purposes

MERS_VFLIP
(SEQ ID NO: 25)
YVDVGPDSVKSACIEVDIQQTFFDKTWPRPIDVSKADGIIYPQGRTYSNITITYQGLFPYQGD
HGDMYVYSAGHATGTTPQKLFVANYSQDVKQFANGFVVRIGAAANSTGTVIISPSTSATIR
KIYPAFMLGSSVGNFSDGKMGRFFNHTLVLLPDGCGTLLRAFYCILEPRSGNHCPAGNSYT
SFATYHTPATDCSDGNYNRNASLNSFKEYFNLRNCTFMYTYNITEDEILEWFGITQTAQGV
HLFSSRYVDLYGGNMFQFATLPVYDTIKYYSIIPHSIRSIQSDRKAWAAFYVYKLQPLTFLL
DFSVDGYIRRAIDCGFNDLSQLHCSYESFDVESGVYSVSSFEAKPSGSVVEQAEGVECDFSP
LLSGTPPQVYNFKRLVFTNCNYNLTKLLSLFSVNDFTCSQISPAAIASNCYSSLILDYFSYPLS
MKSDLSVSSAGPISQFNYKQSFSNPTCLILATVPHNLTTITKPLKYSYINKCSRLLSDDRTEVP
QLVNANQYSPCVSIVPSTVWEDGDYYRKQLSPLEGGGWLVASGSTVAMTEQLQMGFGITV
QYGTDTNSVCPKLEFANDTKIASQLGNCVEYSLYGVSGRGVFQNCTAVGVRQQRFVYDA
YQNLVGYYSDDGNYYCLRACVSVPVSVIYDKETKTHATLFGSVACEHISSTMSQYSRSTRS
MLKRRDSTYGPLQTPVGCVLGLVNSSLFVEDCKLPLGQSLCALPDGGSGGSMRLASIAFN
HPIQVDQLNSSYFKCSIPTNFSFGVTQEYIQTTIQKVTVDCKQYVCNGFQKCEQLLREYGQF
CSKINQALHGANLRQDDSVRNLFASVKSSQSSPIIPGFGGDFNLTLLEPVSISTGSRSARSPIE
DLLFDKVTIADPGYMQGYDDCMQQGPASARDLICAQYVAGYKVLPPLMDVNMEAAYTSS
LLGSIAGVGWTAGLSPFCAIPFPQSIFYRLNGVGITQQVLSENQKLIANKENQALGAMQTGF
TTTPEAFQKVQDAVNNNAQALSKLASELSNTFGAISASIGDIIQRLDVPEQDAQIDRLIYIKGS
GYIPEAPRDGQAYVRKDGEWVLLSTFLLEVLFQGPAGWSHPQFEKGGGSGGGSGGGSWSHPQ
FEK

Bold are the stabilizing mutations (VLIP technology) applied to SARS-CoV-1

Italics are amino acids are exogenous tags for stabilization and purification purposes

HKU1_VFLIP
(SEQ ID NO: 26)
VIGDFNCTNSFINDYNKTIPRISEDVVDVSLGLGTYYVLNRVYLNTTLLFTGYFPKSGANFR
DLALKGSIYLSTLWYKPPFLSDFNNGIFSKVKNTKLYVNNTLYSEFSTIVIGSVFVNTSYTIV
VQPHNGILEITACQYTMCEYPHTVCKSKGSIRNESWHIDSSEPLCLFKKNFTYNVSADWLYF
HFYQERGVFYAYYADVGMPTTFLFSLYLGTILSHYYVMPLTCNAISSNTDNETLEYWVTPL
SRRQYLLNFDEHGVITNAVDCSSSFLSEIQCKTQSFAPNTGVYDLSGFTVKPVATVYRRIPN
LPDCDIDNWLNNVSVPSPLNWERRIFSNCNFNLSTLLRLVHVDSFSCNNLDKSKIFGSCFNSI
TVDKFAIPNRRRDDLQLGSSGFLQSSNYKIDISSSSCQLYYSLPLVNVTINNENPSSWNRRYG
FGSFNLSSYDVVYSDHCFSVNSDFCPCADPSVVNSCAKSKPPSAICPAGTKYRHCDLDTTLY
VKNWCRCSCLPDPISTYSPNTCPQKKVVVGIGEHCPGLGINEEKCGTQLNHSSCFCSPDAFL
GWSFDSCISNNRCNIFSNFIFNGINSGTTCSNDLLYSNTEISTGVCVNYDLYGITGQGIFKEVS
AAYYNNWQNLLYDSNGNIIGFKDFLINKTYTILPCYSGRVSAAFYQNSSSPALLYRNLKCS
YVLNNISFISQPFYFDSYLGCVLNAVNLTSYSVSSCDLRMGSGFCIDYAGGSGGSPYRFVTF
EPFNVSFVNDSCETVGGLFEIQIPTNFTIAGHEEFIQTSSPKVTIDCSAFVCSNYAACHDLLSE
YGTFCDNINSILNEVNDLLDITQLQVANALMQGVTLSSNLNTNLHSDVDNIDFKSLLGCLGS
QCGSSSRSPLEDLLFNKVKLSDVGFVEAYNNCTGGSEIRDLLCVQSFNGIKVLPPILSETQIS
GYTTAATVAAMFPCWSPAAGVPFPLNVQYRINGLGVTMDVLNKNQKLIANAFNKALLSIQ
NGFTATPSALAKIQSVVNANAQALNSLLQQLFNKFGAISSSLQEILSRLDNPEAQVQIDRLIN
GRLTALNAYVSQQLSDITLIKAGASRAIEKVNECVKSQSPRINFCGNGNHILSLVQNAPYGL
LFIHFSYKPTSFKTVLVSPGLCLSGDRGIAPKQGYFIKQNDSWMFTGSSYYYPEPISDKNVVF
MNSCSVNFTKAPFIYLNNSIPNLSDFEAELSLWFKNHTSIAPNLTENSHINATFLDLYYEMNV
IQESIKSLNGSGYIPEAPRDGQAYVRKDGEWVLLSTFLGLEVLFQYIKGSGYIPEAPRDGQAY
VRKDGEWVLLSTFLLEVLFQGPAGWSHPQFEKGGGSGGGSGGGSWSHPQFEK

Bold are the stabilizing mutations (VLIP technology) applied to SARS-CoV-1

Italics are amino acids are exogenous tags for stabilization and purification purposes

OC43_VFLIP
(SEQ ID NO: 27)
MPMGSLQPLATLYLLGMLVASVLAVIGDLKCTSDNINDKDTGPPPISTDTVDVTNGLGTYY
VLDRVYLNTTLFLNGYYPTSGSTYRNMALKGSVLLSRLWFKPPFLSDFINGIFAKVKNTKVI
KDRVMYSEFPAITIGSTFVNTSYSVVVQPRTINSTQDGDNKLQGLLEVSVCQYNMCEYPQTI
CHPNLGNHRKELWHLDTGVVSCLYKRNFTYDVNADYLYFHFYQEGGTFYAYFTDTGVVT
KFLFNVYLGMALSHYYVMPLTCNSKLTLEYWVTPLTSRQYLLAFNQDGIIFNAVDCMSDF
MSEIKCKTQSIAPPTGVYELNGYTVQPIADVYRRKPNLPNCNIEAWLNDKSVPSPLNWERK
TFSNCNFNMSSLMSFIQADSFTCNNIDAAKIYGMCFSSITIDKFAIPNGRKVDLQLGNLGYLQ
SFNYRIDTTATSCQLYYNLPAANVSVSRFNPSTWNKRFGFIEDSVFKPRPAGVLTNHDVVY
AQHCFKAPKNFCPCKLNGSCVGSGPGKNNGIGTCPAGTNYLTCDNLCTPDPITFTGTYKCP
QTKSLVGIGEHCSGLAVKSDYCGGNSCTCRPQAFLGWSADSCLQGDKCNIFANFILHDVNS
GLTCSTDLQKANTDIILGVCVNYDLYGILGQGIFVEVNATYYNSWQNLLYDSNGNLYGFR
DYITNRTFMIRSCYSGRVSAAFHANSSEPALLFRNIKCNYVFNNSLTRQLQPINYFDSYLGC
VVNAYNSTAISVQTCDLTVGSGYCVDYSGGSGGSGYRFTNFEPFTVNSVNDSCEPVGGLY
EIQIPSEFTIGNMVEFIQTSSPKVTIDCAAFVCGDYAACKSQLVEYGSFCDNINAILTEVNELL
DTTQLQVANSLMNGVTLSTKLKDGVNFNVDDINFSPVLGCLGSECSKASSRSPIEDLLFDK
VKLSDVGFVEAYNNCTGGAEIRDLICVQSYKGIKVLPPLLSENQFSGYTLAATSASLFCPWT
PAAGVPFPLNVQYRINGLGVTMDVLSQNQKLIANAFNNALYAIQEGFDATPSALVKIQAVV
NANAEALNNLLQQLSNRFGAISASLQEILSRLDAPEAEAQIDRLINGRLTALNAYVSQQLSD
STLVKFSAAQAMEKVNECVKSQSSRINFCGNGNHIISLVQNAPYGLYFIHFSYVPTKYVTAR
VSPGLCIAGDRGIAPKSGYFVNVNNTWMYTGSGYYYPEPITENNVVVMSTCAVNYTKAPY
VMLNTSIPNLPDFKEELDQWFKNQTSVAPDLSLDYINVTFLDLLIKRMKQIEDKIEEIESKQK
KIENEIARIKKIKLVPRGSLEWSHPQFEK

Bold are the stabilizing mutations (VLIP technology) applied to OC43

CHIMERIC PROTEINS; GRAFTING SARS1, SARS2 AND MERS ONTO VFLIP SCAFFOLD

VFLIP_RBD:SARS1_HT-A
(SEQ ID NO: 28)
QCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCN
DPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEKGIYQTSNFRVQ
PTESIVRFPNITNLCPFGEVENATKFPSVYAWERKKISNCVADYSVLYNSTFFSTFKCYGVSA
TKLNDLCFSNVYADSFVVKGDDVRQIAPGQTGVIADYNYKLPDDFMGCVLAWNTRNIDA
TSTGNYNYKYRYLRHGKLRPFERDISNVPFSPDGKPCTPPALNCYWPLNDYGFYTTTGIGY
QPYRVVVLSFELLNAPATVCGPKLGKIADYNYKLPDDFTGCVIAWNSNNLDSKVGGNYNY
LYRLFRKSNLKPFERDISTEIYQAGSTPCNGVEGENCYFPLQSYGFQPTNGVGYQPYRVVVL
SFELLHAPATVCGPKKSTNLVKNKCVNFNFNGLTGTGVLTESNKKFLPFQQFGRDIADTTD
AVRDPQTLEILDITPCSFGGVSVITPGTNTSNQVAVLYQGVNCTEVPVAIHADQLTPTWRVY
STGSNVFQTRAGCLIGAEHVNNSYECDIPIGAGICASYQGGSGGSSIIAYTMSLGAENSVACS
NNSIAIPTNFTISVTTEILPVSMTKTSVDCTMYICGDSTECSNLLLQYGSFCTQLNRALTGIAV
EQDKNTQEVFAQVKQIYKTPPIKDFGGFNFSQILPDPSKPSKRSPIEDLLFNKVTLADAGFIK
QYGDCLGDIAARDLICAQKFNGLTVLPPLLTDEMIAQYTSALLAGTICSGWTFGAGPALQIP
FPMQMAYRFNGIGVTQNVLYENQKLIANQFNSAIGKIQDSLSSTPSALGKLQDVVNQNAQA
LNTLVKQLSSNFGAISSVLNDILSRLDKPEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQELGK
YEQYIKGSGSASGAEIAAIEYEQAAIKEEIAAIKDKIAAIKEYIAAI
VFLIP_RBD:SARS2_HT-B
(SEQ ID NO: 29)
QCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCN
DPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEKGIYQTSNFRVQ
PTESIVRFPNITNLCPFGEVENATRFASVYAWNRKRISNCVADYSVLYNSASFSTFKCYGVS
PTKLNDLCFTNVYADSFVIRGDEVRQIAPGQTGKIADYNYKLPDDFTGCVIAWNSNNLDSK
VGGNYNYLYRLFRKSNLKPFERDISTEIYQAGSTPCNGVEGENCYFPLQSYGFQPTNGVGY
QPYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQDVNCTEVPVAIHADQ
LTPTWRVYSTGSNVFQTRAGCLIGAEHVNNSYECDIPIGAGICASYQGGSGGSSIIAYTMSL
GAENSVACSNNSIAIPTNFTISVTTEILPVSMTKTSVDCTMYICGDSTECSNLLLQYGSFCTQ
LNRALTGIAVEQDKNTQEVFAQVKQIYKTPPIKDFGGFNFSQILPDPSKPSKRSPIEDLLENK
VTLADAGFIKQYGDCLGDIAARDLICAQKFNGLTVLPPLLTDEMIAQYTSALLAGTICSGW
TFGAGPALQIPFPMQMAYRFNGIGVTQNVLYENQKLIANQFNSAIGKIQDSLSSTPSALGKL
QDVVNQNAQALNTLVKQLSSNFGAISSVLNDILSRLDKPEAEVQIDRLITGRLQSLQTYVTQ
QLIRAAEIRASANLAATKMSECVLGQSKRVDFCGKGYHLMSFPQSAPHGVVFLHVTYVPA
QEKNFTTAPAICHDGKAHFPREGVFVSNGTHWFVTQRNFYEPQIITTDNTFVSGNCDVVIGI
VNNTVYDPLQPELDSFKEELDKYFKNHTSPDVDLGDISGINASVVNIQKEIDRLNEVAKNLN
ESLIDLQELGKYEQGSASGEKIAAIKEEQAAIEEEIQAIKEEIAAIKYLIAQI

Bold are the stabilizing mutations (VLIP technology) applied to RBD:SARS2_HT-B

VFLIP_RBD:MERS_HT-C
(SEQ ID NO: 30)
QCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCN
DPFLGVYYHKNNKSWMESEFRVYSSANNCTFEYVSQPFLMDLEGKQGNFKNLREFVFKNI
DGYFKIYSKHTPINLVRDLPQGFSALEPLVDLPIGINITRFQTLLALHRSYLTPGDSSSGWTA
GAAAYYVGYLQPRTFLLKYNENGTITDAVDCALDPLSETKCTLKSFTVEKGIYQTSNFRVQ
PTESIVRFPGVECDFSPLLSGTPPQVYNFKRLVFTNCNYNLTKLLSLFSVNDFTCSQISPAAIA
SNCYSSLILDYFSYPLSMKSDLSVSSAGPISQFNYKQSFSNPTCLILATVPHNLTTITKPLKYS
YINKCSRLLSDDRTEVPQLVNANQYSPCVSIVPSTVWEDGDYYRKQLSPLEGGGWLVASGS
TVAMTEQLQMGFGITVQYGTDTNSVCPKLGKIADYNYKLPDDFTGCVIAWNSNNLDSKVG
GNYNYLYRLFRKSNLKPFERDISTEIYQAGSTPCNGVEGFNCYFPLQSYGFQPTNGVGYQP
YRVVVLSFELLHAPATVCGPKKSTNLVKNKCVNFNFNGLTGTGVLTESNKKFLPFQQFGR
DIADTTDAVRDPQTLEILDITPCSFGGVSVITPGTNTSNQVAVLYQGVNCTEVPVAIHADQL
TPTWRVYSTGSNVFQTRAGCLIGAEHVNNSYECDIPIGAGICASYQGGSGGSSIIAYTMSLG
AENSVACSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSPIEDLLFNKV
TLADAGFIKQYGDCLGDIAARDLICAQKFNGLTVLPPLLTDEMIAQYTSALLAGTICSGWTF
GAGPALQIPFPMQMAYRFNGIGVTQNVLYENQKLIANQFNSAIGKIQDSLSSTPSALGKLQD
VVNQNAQALNTLVKQLSSNFGAISSVLNDILSRLDKPEAEVQIDRLITGRLQSLQTYVTQQL
IRAAEIRASANLAATKMSECVLGQSKRVDFCGKGYHLMSFPQSAPHGVVFLHVTYVPAQE
KNFTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLNES
LIDLQELGKYEQYIKGSGSASGAEIAAIKYKQAAIKNEIAAIKQEIAAIEQMIAAI

The recombinant spikes can be produced in transiently transfected ExpiCHO cells and the urified protein evaluated for homogeneity and stability by both CG-MALS and differential scanning calorimetry. Spike binding to neutralizing antibodies and recombinant receptors (e.g., ACE2 for SARS, DPP4 for MERS) can be assessed by ELISA and examine the structural shape and homogeneity of the modified spikes using, e.g., Titan electron microscopes. Newly engineered VFLIP hCoV spikes that have ideal shape, stability, and reactivity will be tested for evaluation of immunogenicity and the type and quality of antibodies elicited. The improved designs are expected to elicit better antibodies against the conserved regions of S2 that require proper or native quaternary assembly of the multiple 51 and S2 copies in the trimer. The improved immunogens are expected to neutralizing over non-neutralizing antibodies in these regions than current S-2P-based antigens. Notably, use of viral surface glycoproteins engineered to remain in their proper prefusion, oligomeric assemblies has frequently led to identification and elicitation of novel antibody responses that cannot be attained with less stable immunogens.

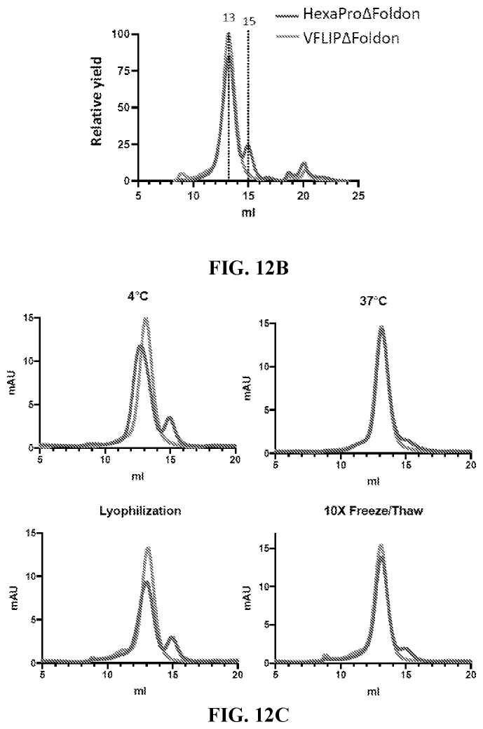

FIGS. 12A to 12C show: FIG. 12A shows that VFLIP is more thermostable than HexaPro, with 3° C. higher Tm. FIG. 12B shows that VFLIP retains its trimeric structure even after removal of the Foldon trimerization domain (VFLIPΔFoldon). FIG. 12C shows that VFLIPΔFoldon remains trimeric after lyophilization, multiple freeze/thaw cycles, and prolonged storage at either 4° C. or at room temperature.

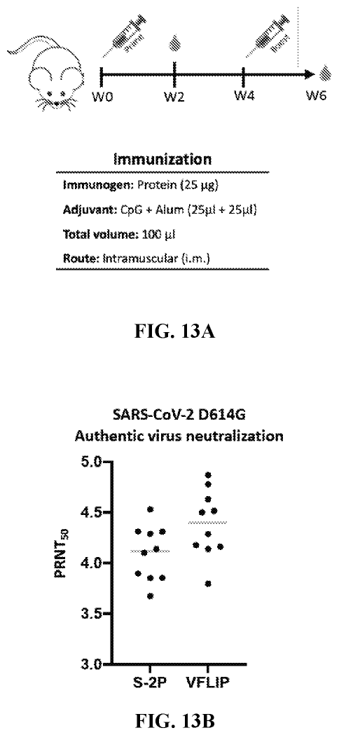

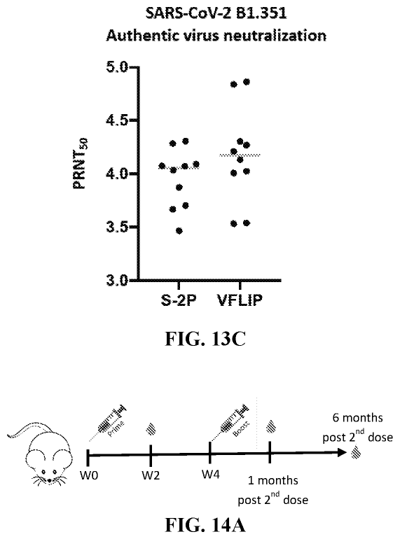

FIGS. 13A to 13C show: FIG. 13A the immunization schedule and dosage, and assays using authentic D614G (FIG. 13B) and B.1.351 (FIG. 13C) SARS-CoV-2 showed that VFLIP-induced sera had a higher neutralizing potency compared to S-2P, with 50% neutralization at dilutions of 1:30,000 and 1:13,000, respectively.

Sequences include Foldon and StrepTags in italics. Variant mutations are in bold, and VFLIP features in underline (including hGluc SP). P681R is not present as it is replaced by GGSGGS (SEQ ID NO:36) linker.

B.1.617.1 (Kappa) VFLIP:.
(SEQ ID NO: 31)
MGVKVLFALICIAVAEAQCVNLTTRTQLPPAYTNSFTRDVYYPDKVFRSSVLHSTQ
DLFLPFFSNVTWFHAIHVSGTNGTKRFDNPVLPFNDGVYFASIEKSNIIRGWIFGTTLDSKTQ
SLLIVNNATNVVIKVCEFQFCNDPFLDVYYHKNNKSWMKSEFRVYSSANNCTFEYVSQPFL
MDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGKIADYNYK
LPDDFTGCVIAWNSNNLDSKVGGNYNYRYRLFRKSNLKPFERDISTEIYQAGSTPCNGVQG
FNCYFPLQSYGFQPTNGVGYQPYRVVVLSFELLHAPATVCGPKKSTNLVKNKCVNFNFNG
LTGTGVLTESNKKFLPFQQFGRDIADTTDAVRDPQTLEILDITPCSFGGVSVITPGTNTSNQV
AVLYQGVNCTEVPVAIHADQLTPTWRVYSTGSNVFQTRAGCLIGAEHVNNSYECDIPIGAG
ICASYQGGSGGSSIIAYTMSLGAENSVACSNNSIAIPTNFTISVTTEILPVSMTKTSVDCTMYI
CGDSTECSNLLLQYGSFCTQLNRALTGIAVEQDKNTQEVFAQVKQIYKTPPIKDFGGFNFSQ
ILPDPSKPSKRSPIEDLLFNKVTLADAGFIKQYGDCLGDIAARDLICAQKFNGLTVLPPLLTD
EMIAQYTSALLAGTICSGWTFGAGPALQIPFPMQMAYRENGIGVTQNVLYENQKLIANQFN
SAIGKIQDSLSSTPSALGKLQDVVNQNAQALNTLVKQLSSNFGAISSVLNDILSRLDKPEAE
VQIDRLITGRLQSLQTYVTQQLIRAAEIRASANLAATKMSECVLGQSKRVDFCGKGYHLMS
FPQSAPHGVVFLHVTYVPAHEKNFTTAPAICHDGKAHFPREGVFVSNGTHWFVTQRNFYE
PQIITTDNTFVSGNCDVVIGIVNNTVYDPLQPELDSFKEELDKYFKNHTSPDVDLGDISGINA
SVVNIQKEIDRLNEVAKNLNESLIDLQELGKYEQYIKGSGYIPEAPRDGQAYVRKDGEWVLLS
TFLLEVLFQGPAGWSHPQFEKGGGSGGGSGGGSWSHPQFEK
B.1.617.2 (Delta) VFLIP:.
(SEQ ID NO: 32)
MGVKVLFALICIAVAEAQCVNLRTRTQLPPAYTNSFTRDVYYPDKVFRSSVLHSTQ
DLFLPFFSNVTWFHAIHVSGTNGTKRFDNPVLPFNDGVYFASTEKSNIIRGWIFGTTLDSKT
QSLLIVNNATNVVIKVCEFQFCNDPFLGVYYHKNNKSWMES--
GVYSSANNCTFEYVSQPFLMDLEGKQGNFKNLREFVFKNIDGYFKIYSKHTPINL VRDLPQ
GFSALEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGEVEN
ATRFASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYRYRLFRKSNLKPF
ERDISTEIYQAGSKPCNGVEGENCYFPLQSYGFQPTNGVGYQPYRVVVLSFELLHAPATVC
GPKKSTNLVKNKCVNFNFNGLTGTGVLTESNKKFLPFQQFGRDIADTTDAVRDPQTLEILDI
TPCSFGGVSVITPGTNTSNQVAVLYQGVNCTEVPVAIHADQLTPTWRVYSTGSNVFQTRAG
CLIGAEHVNNSYECDIPIGAGICASYQGGSGGSSIIAYTMSLGAENSVACSNNSIAIPTNFTIS
VTTEILPVSMTKTSVDCTMYICGDSTECSNLLLQYGSFCTQLNRALTGIAVEQDKNTQEVFA
QVKQIYKTPPIKDFGGFNFSQILPDPSKPSKRSPIEDLLFNKVTLADAGFIKQYGDCLGDIAA
RDLICAQKFNGLTVLPPLLTDEMIAQYTSALLAGTICSGWTFGAGPALQIPFPMQMAYRFN
GIGVTQNVLYENQKLIANQFNSAIGKIQDSLSSTPSALGKLQNVVNQNAQALNTLVKQLSS
NFGAISSVLNDILSRLDKPEAEVQIDRLITGRLQSLQTYVTQQLIRAAEIRASANLAATKMSE
CVLGQSKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKGSGYI
PEAPRDGQAYVRKDGEWVLLSTELLEVLFQGPAGWSHPQFEKGGGSGGGSGGGSWSHPQFEK
Mutations and the amino acid sequence of VFLIP_Lambda, VFLIP:
(SEQ ID NO: 33): G75V, T76I, Δ246-252, L452Q, F490S, D614G and T859N.
QCVNLTTRTQLPPAYTNSFTRGVYYPDKVFRSSVLHSTQDLFLPFFSNVTWFHAIH
VSGTNVIKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNFKNLREF
VFKNIDGYFKIYSKHTPINLVRDLPQGFSALEPLVDLPIGINITRFQTLLALHDSSSGWTAGA
AAYYVGYLQPRTFLLKYNENGTITDAVDCALDPLSETKCTLKSFTVEKGIYQTSNFRVQPT
ESIVRFPNITNLCPFGEVENATRFASVYAWNRKRISNCVADYSVLYNSASFSTFKCYGVSPT
KLNDLCFTNVYADSFVIRGDEVRQIAPGQTGKIADYNYKLPDDFTGCVIAWNSNNLDSKV
GGNYNYQYRLFRKSNLKPFERDISTEIYQAGSTPCNGVEGFNCYSPLQSYGFQPTNGVGYQ
PYRVVVLSFELLHAPATVCGPKKSTNLVKNKCVNFNFNGLTGTGVLTESNKKFLPFQQFGR
DIADTTDAVRDPQTLEILDITPCSFGGVSVITPGTNTSNQVAVLYQGVNCTEVPVAIHADQL
TPTWRVYSTGSNVFQTRAGCLIGAEHVNNSYECDIPIGAGICASYQGGSGGSSIIAYTMSLG
AENSVACSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSPIEDLLFNKV
TLADAGFIKQYGDCLGDIAARDLICAQKFNGLNVLPPLLTDEMIAQYTSALLAGTICSGWTF
GAGPALQIPFPMQMAYRFNGIGVTQNVLYENQKLIANQFNSAIGKIQDSLSSTPSALGKLQD
VVNQNAQALNTLVKQLSSNFGAISSVLNDILSRLDKPEAEVQIDRLITGRLQSLQTYVTQQL
IRAAEIRASANLAATKMSECVLGQSKRVDFCGKGYHLMSFPQSAPHGVVFLHVTYVPAQE
KNFTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLNES
LIDLQELGKYEQ

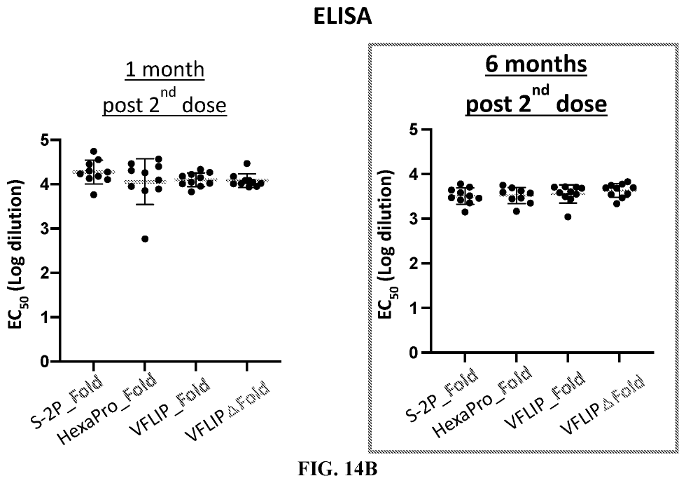

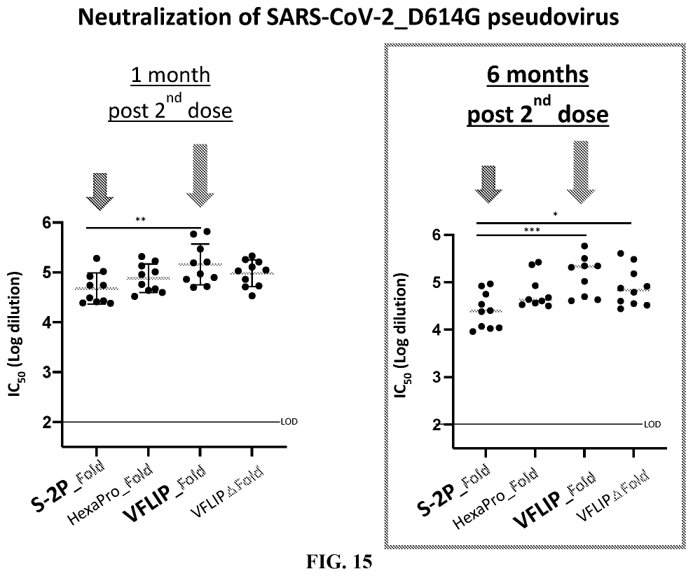

VFLIP elicits potently neutralizing responses in immunized mice. The inventors hypothesized that the additional stability afforded by VFLIP would improve elicitation of neutralizing antibodies and that removing Foldon (VFLIPΔFoldon) would avoid deleterious responses to this exogenous trimerization domain. To assess the immunogenicity of VFLIP, BALB/c mice were immunized with four versions of spike: (1) Parental S-2P, (2) HexaPro, (3) VFLIP, and (4) VFLIPΔFoldon. Overall, mice in all four groups mounted robust antibody responses as evidenced by total anti-spike antibody titers (FIGS. 14A, 14B) with no statistical differences in the antibody titers among the different groups and across the two different time points tested (1 month and 6 months post second dose). FIG. 14B shows that sera from immunized mice were used to examine activity in neutralization assays using rVSV-pseudotyped with SARS-CoV-2 spike bearing the D614G mutation, as well as authentic SARS-CoV-2 bearing the D614G substitution and authentic SARS-CoV-2 virus of the B.1.351 (South African) lineage, which is a current variant of concern. It was found that binding looks the same for binding to whole spike anywhere after the immunizations, however, VFLIP without foldon (trimerization domain can be removed after the protein is formed, not required to hold things together like in 2P or Hexapro—fraction of these fall apart into monomers if the trimerization domain is removed). Several of the SARS-CoV-2 vaccines that are currently available on the market (including Pfizer, Inc./BioNTech's BNT162b2/“COMIRNATY” and Moderna's mRNA-1273/“Spikevax” SARS-CoV-2 vaccines) use 2P.

Pseudovirus neutralization titers for VFLIP-immunized sera were significantly higher than S-2P and achieved 50% neutralization at dilutions over 1:100,000 in the samples collected one months after the second dose (FIG. 15). More interestingly, when measured the long-lasting neutralizing activity in sera 6 months after the second dose, VFLIP and VFLIPΔFoldon showed the best neutralizing activity with significant differences with S-2P, and also showed a better neutralizing antibody titers than HexaPro in both measured time points.

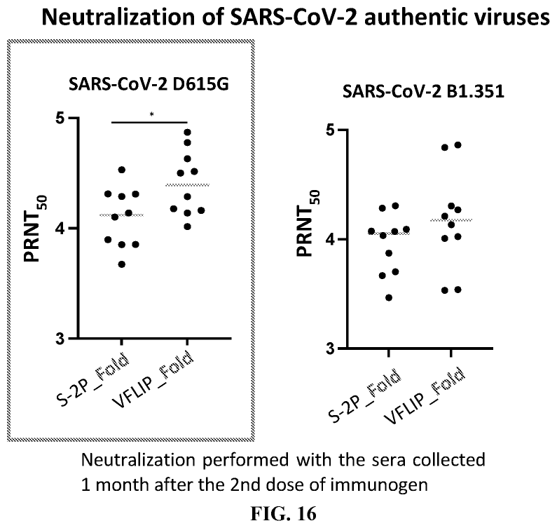

Assays using authentic D614G and B.1.351 showed that VFLIP-induced sera had a higher neutralizing potency compared to S-2P, with 50% neutralization at dilutions of 1:30,000 and 1:13,000, respectively (FIG. 16). Pseudovirus neutralization of VFLIP-ΔFoldon (with trimerization domain removed) was equivalent to that for Foldon-containing HexaPro, indicating that immunogenicity is maintained without an exogenous trimerization motif. VFLIP neutralizes better authentic life SARS-CoV-2_D614G virus than S-2P, with the same with other variants (South Africa). This figure demonstrates that with both VFLIP and VFLIPΔFoldon more neutralizing antibodies were obtained than with an S-2P immunization at 1 and 6 months, which improvement was statistically significant.

Together, these data demonstrate that VFLIP is a robust immunogen capable of inducing a strong antibody response able to neutralize both authentic and pseudotyped SARS-CoV2 against the parental variant (D614G) as well as other SARS-CoV2 variants of concern.

The stability of VFLIP, its robust production, and its ability to elicit a potent long-lasting antibody response capable of neutralizing the infectivity of different variants of SARS-CoV-2, as aspects of the present disclosure describe, make this immunogen ideal for diagnostics, therapeutics, and novel coronavirus vaccines including pan-coronavirus vaccine embodiments.

The biophysical characteristics of VFLIP allow its further study in novel vaccine platforms such mRNA and nanoparticle-based vaccines, which, as aspects of the present disclosure describe, allows for the design of broader and more potent pan-coronavirus vaccines.

### Example 2

### VFLIP Variants Delivered in Protein Nanoparticles, e.g., Ferritin Nanoparticles (VFLIP_Fr_NPs): a Self-Assembling One-Component Gene Strategy

Multimeric presentation of antigen can elicit responses that are superior to recombinant subunit vaccines alone. Moreover, multivalent antigens more potently activate naive B cells via avidity and ability to cross-link BCRs that in turn can elicit more promiscuous, cross-reactive antibodies that have lower germline affinities. To compare the activity of soluble subunit immunogens to that of multimeric presentation produced from the same construct, Ferritin nanoparticles displaying VFLIP SARS-CoV-2 or spikes are prepared from other CoVs stabilized with the VFLIP approach. Self-assembling protein nanoparticles (SAPN) are multimeric particles such as ferritin or lumazine synthase that can be exploited for multivalent display of viral antigens using chemical linkage, direct gene fusion, or ligand:ligand interactions, (e.g., SpyTag/SpyCatcher system). To assess how multivalent display affects immunogenicity, two nanoparticle platforms: 24-mer ferritin and 60-mer 13-01 are tested. Ferritin nanoparticles have been widely used as candidate nanoparticles for HIV, HCV, and influenza vaccines whereas 13-01, a computationally designed hyperstable nanocage, has shown promise for HIV, HCV, and RSV. Both platforms have been used in pre-clinical SARS-CoV-2 vaccine development, and in animal studies these nanoparticles demonstrated superior immunogenicity, particularly in eliciting broad anti-CoV responses. ELISA can be used to evaluate antibody and receptor binding of multimeric versions of spike and electron microscopy to probe structural stability and epitope presentation. Many near-germline antibodies against SARS-CoV-2 have good affinity with little somatic hypermutation. When numerous “on-target” naive B cells have high germline affinity, a lower-valency vaccine immunogen could avoid extensive engagement of “off-target” sites. As such, we will first prime with 60-mer (20 spike trimers) nanoparticles and boost with 24-mer (8 spike trimers), or immunize only with 24-mer.

VFLIP variants will be genetically fused to the heavy chain of the human ferritin protein, linking them with a Gly-Ser that can differ in length depending on the antigen used. VFLIP with and without foldon are tested to determine which of the constructs provide more yield and stability of the resulting VFLIP_Fr_NP. The ORF will be flanked by a purification tag (double tween streptavidin tag) followed by an enterokinase (EK) cleavage site, both upstream (5′ of the gene).

VFLIP_Fr_NPs can be expressed in ExpiCHO cells using the manufacturer's “High Titer” protocol with a 7-day culture incubation to assess relative expression. Briefly, plasmid DNA and Expifectamine are mixed in Opti-PRO SFM (Gibco) according to the manufacturer's instructions, and added to the cells. On day 1, cells are fed with manufacturer-supplied feed and enhancer as specified in the manufacturer's protocol, and cultures were moved to a shaker incubator set to 32° C., 5% CO2 and 115 RPM. On day 7, the cultures were clarified by centrifugation, BioLock was added, and supernatants are passed through a 0.22 μM sterile filter. Two-steps purification will be carried out. First, an affinity chromatography purification on an ÄKTA go system (Cytivia) using a 5 mL StrepTrap-HP column equilibrated with TBS buffer (25 mM Tris pH 7.6, 200mM NaCl, 0.02% NaN3), and eluted in TBS buffer supplemented with 5 mM d-desthiobiotin (Sigma Aldrich). A second-step performed by size-exclusion-chromatography (SEC) on a Superdex 6 Increase 10/300 column (Cytivia) in the same TBS buffer.

The resulting VFLIP Fr NPs are validated by negative staining electron microscopy (nsEM), Differential Scanning calorimetry (DLS) and conventional SDS-PAGEs. Antigenicity of the NPs will be tested with several SARS-CoV-2 monoclonal antibodies.

### Example 3

### Production of Chimeric Spike Proteins by Using VFLIP Technology

Due to its low sequence conservation, antibodies that bind multiple CoV species usually bind outside the RBD, whereas antibodies that target the RBD tend to be SARS-CoV-2-specific or cross-react only with SARS-CoV. Anti-RBD mAbs, however, are the most likely to neutralize infectious virus. Moreover, the RBD is highly immunogenic and is the target of most neutralizing antibodies (and the most potent neutralizing antibodies) that can be detected using current probes and immunogens. Using VFLIP, we are performing another strategy for eliciting cross-reactive immunity involved improving display of cross-reactive epitopes on other regions of spike by stabilizing the more conserved S2 subunit and core of spike from SARS-CoV-2 and other relevant CoVs. An ideal response could include antibodies that are themselves cross-reactive. Spike immunogens generated were fully of one species. We are testing SARS2 core+SARS1 RBD or MERS core+SARS2 RBD, and all possible combinatory including all current human-infective circulating CoVs. Delivery of multiple spikes in a cocktail, or delivered singly but sequentially in a prime/boost regimen could elicit cross-reactive antibodies.

A complementary strategy accepts that antibodies against the RBD are less likely to be cross-reactive, yet are still desirable. Cross-reactivity toward the RBD may instead come collectively in a polyclonal response. The inventors' design a single, chimeric immunogen in which the successful SARS-CoV-2 VFLIP core trimer is linked with RBDs from three separate CoVs, and chimerically trimerized using a heterotrimeric coiled-coil motif protein (PDB:1BB1). This chimera retains the conserved core structure but displays different RBDs—in an example embodiment not intended to limit the disclosure, one each from SARS-CoV-2, SARS-CoV and MERS, and each flexibly linked to the VFLIP core. This strategy is also applied to obtain VFLIP proteins with three different monomers from three different SARS-CoV-2 variants of concern (i.e., VFLIP trimer formed with 3 monomers belonged to Parental Wuhan D614G sequence, Beta and Delta VOC). This strategy is applicable to all SARS-CoV-2 VOC circulating and all the new VOC that may arise. The structure of the trimer and conserved quaternary epitopes would remain stable as they are derived entirely SARS-CoV-2 and thus preserve the natively glycosylated VFLIP core, for which the inventors already have a high-resolution structure.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Afonine, Pavel V., Billy K. Poon, Randy J. Read, Oleg V. Sobolev, Thomas C. Terwilliger, Alexandre Urzhumtsev, and Paul D. Adams. 2018. “Real-Space Refinement in PHENIX for Cryo-EM and Crystallography.” Acta Crystallographica. Section D, Structural Biology 74 (Pt 6): 531-44.

Benton, Donald J., Antoni G. Wrobel, Pengqi Xu, Chloe Roustan, Stephen R. Martin, Peter B. Rosenthal, John J. Skehel, and Steven J. Gamblin. 2020. “Receptor Binding and Priming of the Spike Protein of SARS-CoV-2 for Membrane Fusion.” Nature 588 (7837): 327-30.

Blundell, Patricia A., Dongli Lu, Anne Dell, Stuart Haslam, and Richard J. Pleass. 2020. “Choice of Host Cell Line Is Essential for the Functional Glycosylation of the Fc Region of Human IgG1 Inhibitors of Influenza B Viruses.” Journal of Immunology 204 (4): 1022-34.

Bos, Rinke, Lucy Rutten, Joan E. M. van der Lubbe, Mark J. G. Bakkers, Gijs Hardenberg, Frank Wegmann, David Zuijdgeest, et al. 2020. “Ad26 Vector-Based COVID-19 Vaccine Encoding a Prefusion-Stabilized SARS-CoV-2 Spike Immunogen Induces Potent Humoral and Cellular Immune Responses.” NPJ Vaccines 5: 91.

Brouwer, Philip J. M., Tom G. Caniels, Karlijn van der Straten, Jonne L. Snitselaar, Yoann Aldon, Sandhya Bangaru, Jonathan L. Tones, et al. 2020. “Potent Neutralizing Antibodies from COVID-19 Patients Define Multiple Targets of Vulnerability.” Science 369 (6504): 643-50.

Cai, Yongfei, Jun Zhang, Tianshu Xiao, Hanqin Peng, Sarah M. Sterling, Richard M. Walsh Jr, Shaun Rawson, Sophia Rits-Volloch, and Bing Chen. 2020. “Distinct Conformational States of SARS-CoV-2 Spike Protein.” Science 369 (6511): 1586-92.

Cao, Liwei, Jolene K. Diedrich, Daniel W. Kulp, Matthias Pauthner, Lin He, Sung-Kyu Robin Park, Devin Sok, et al. 2017. “Global Site-Specific N-Glycosylation Analysis of HIV Envelope Glycoprotein.” Nature Communications 8: 14954.

Casalino, Lorenzo, Zied Gaieb, Jory A. Goldsmith, Christy K. Hjorth, Abigail C. Dommer, Aoife M. Harbison, Carl A. Fogarty, et al. 2020. “Beyond Shielding: The Roles of Glycans in the SARS-CoV-2 Spike Protein.” ACS Central Science 6 (10): 1722-34.

Cirelli, Kimberly M., and Shane Crotty. 2017. “Germinal Center Enhancement by Extended Antigen Availability.” Current Opinion in Immunology 47: 64-69.

Corbett, Kizzmekia S., Darin K. Edwards, Sarah R. Leist, Olubukola M. Abiona, Seyhan Boyoglu-Barnum, Rebecca A. Gillespie, Sunny Himansu, et al. 2020. “SARS-CoV-2 mRNA Vaccine Design Enabled by Prototype Pathogen Preparedness.” Nature 586 (7830): 567-71.

Doria-Rose, Nicole A., and M. Gordon Joyce. 2015. “Strategies to Guide the Antibody Affinity Maturation Process.” Current Opinion in Virology 11 (April): 137-47.

Duan, Liangwei, Qianqian Zheng, Hongxia Zhang, Yuna Niu, Yunwei Lou, and Hui Wang. 2020. “The SARS-CoV-2 Spike Glycoprotein Biosynthesis, Structure, Function, and Antigenicity: Implications for the Design of Spike-Based Vaccine Immunogens.” Frontiers in Immunology 11: 576622.

Edwards, Robert J., Katayoun Mansouri, Victoria Stalls, Kartik Manne, Brian Watts, Rob Parks, Sophie M. C. Gobeil, et al. 2021. “Cold Sensitivity of the SARS-CoV-2 Spike Ectodomain.” Nature Structural & Molecular Biology. 28,128-131.

El Shikh, Mohey Eldin M., Rania M. El Sayed, Selvakumar Sukumar, Andras K. Szakal, and John G. Tew. 2010. “Activation of B Cells by Antigens on Follicular Dendritic Cells.” Trends in Immunology 31 (6): 205-11.

Emsley, P., B. Lohkamp, W. G. Scott, and K. Cowtan. 2010. “Features and Development of Coot.” Acta Crystallographica. Section D, Biological Crystallography 66 (Pt 4): 486-501.

“Evaluate Safety and Pharmacokinetics of HLX70 in Healthy Adult Volunteers.” n.d. Accessed Apr. 30, 2021. https://clinicaltrials.gov/ct2/show/NCT04561076.

“Evaluate the Safety, Tolerability, Pharmacodynamics, Pharmacokinetics, and Immunogenicity of HLX71 (Recombinant Human Angiotensin-Converting Enzyme 2-Fc Fusion Protein for COVID-19) in Healthy Adult Subjects.” n.d. Accessed Apr. 30, 2021. https://clinicaltrials.gov/ct2/show/NCT04583228.

Feng, Yu, Karen Tran, Shridhar Bale, Shailendra Kumar, Javier Guenaga, Richard Wilson, Natalia de Val, et al. 2016. “Thermostability of Well-Ordered HIV Spikes Correlates with the Elicitation of Autologous Tier 2 Neutralizing Antibodies.” PLoS Pathogens 12 (8): e1005767.

Finney, Joel, Chen-Hao Yeh, Garnett Kelsoe, and Masayuki Kuraoka. 2018. “Germinal Center Responses to Complex Antigens.” Immunological Reviews 284 (1): 42-50.

Frese, Christian K., A. F. Maarten Altelaar, Henk van den Toorn, Dirk Nolting, Jens Griep-Raming, Albert J. R. Heck, and Shabaz Mohammed. 2012. “Toward Full Peptide Sequence Coverage by Dual Fragmentation Combining Electron-Transfer and Higher-Energy Collision Dissociation Tandem Mass Spectrometry.” Analytical Chemistry 84 (22): 9668-73.

Frese, Christian K., Houjiang Zhou, Thomas Taus, A. F. Maarten Altelaar, Karl Mechtler, Albert J. R. Heck, and Shabaz Mohammed. 2013. “Unambiguous Phosphosite Localization Using Electron-Transfer/higher-Energy Collision Dissociation (EThcD).” Journal of Proteome Research 12 (3): 1520-25.

Ge, Jiwan, Ruoke Wang, Bin Ju, Qi Zhang, Jing Sun, Peng Chen, Senyan Zhang, et al. 2021. “Antibody Neutralization of SARS-CoV-2 through ACE2 Receptor Mimicry.” Nature Communications 12 (1): 250.

Gobeil, Sophie M-C, Katarzyna Janowska, Shana McDowell, Katayoun Mansouri, Robert Parks, Kartik Manne, Victoria Stalls, et al. 2021. “D614G Mutation Alters SARS-CoV-2 Spike Conformation and Enhances Protease Cleavage at the S1/S2 Junction.” Cell Reports 34 (2): 108630.

Goddard, Thomas D., Conrad C. Huang, Elaine C. Meng, Eric F. Pettersen, Gregory S. Couch, John H. Morris, and Thomas E. Ferrin. 2018. “UCSF ChimeraX: Meeting Modern Challenges in Visualization and Analysis.” Protein Science: A Publication of the Protein Society 27 (1): 14-25.

Grant, Oliver C., David Montgomery, Keigo Ito, and Robert J. Woods. 2020. “Analysis of the SARS-CoV-2 Spike Protein Glycan Shield Reveals Implications for Immune Recognition.” Scientific Reports 10 (1): 14991.

Güthe, Sarah, Larisa Kapinos, Andreas Möglich, Sebastian Meier, Stephan Grzesiek, and Thomas Kiefhaber. 2004. “Very Fast Folding and Association of a Trimerization Domain from Bacteriophage T4 Fibritin.” Journal of Molecular Biology 337 (4): 905-15.

Hastie, Kathryn M., Michelle A. Zandonatti, Lara M. Kleinfelter, Megan L. Heinrich, Megan M. Rowland, Kartik Chandran, Luis M. Branco, James E. Robinson, Robert F. Garry, and Erica Ollmann Saphire. 2017. “Structural Basis for Antibody-Mediated Neutralization of Lassa Virus.” Science 356 (6341): 923-28.

He, Linling, Sonu Kumar, Joel D. Allen, Deli Huang, Xiaohe Lin, Colin J. Mann, Karen L. Saye-Francisco, et al. 2018. “HIV-1 Vaccine Design through Minimizing Envelope Metastability.” Science Advances 4 (11): eaau6769.

Henderson, Rory, Robert J. Edwards, Katayoun Mansouri, Katarzyna Janowska, Victoria Stalls, Sophie M. C. Gobeil, Megan Kopp, et al. 2020. “Controlling the SARS-CoV-2 Spike Glycoprotein Conformation.” Nature Structural & Molecular Biology 27 (10): 925-33.

Hsieh, Ching-Lin, Jory A. Goldsmith, Jeffrey M. Schaub, Andrea M. DiVenere, Hung-Che Kuo, Kamyab Javanmardi, Kevin C. Le, et al. 2020. “Structure-Based Design of Prefusion-Stabilized SARS-CoV-2 Spikes.” Science 369 (6510): 1501-5.

Huo, Jiandong, Yuguang Zhao, Jingshan Ren, Daming Zhou, Helen M. E. Duyvesteyn, Helen M. Ginn, Loic Carrique, et al. 2020. “Neutralization of SARS-CoV-2 by Destruction of the Prefusion Spike.” Cell Host & Microbe 28 (3): 445-54.e6.

Juraszek, Jarek, Lucy Rutten, Sven Blokland, Pascale Bouchier, Richard Voorzaat, Tina Ritschel, Mark J. G. Bakkers, Ludovic L. R. Renault, and Johannes P. M. Langedijk. 2021. “Stabilizing the Closed SARS-CoV-2 Spike Trimer.” Nature Communications 12 (1): 244.

Karlsson Hedestam, Gunilla B., Javier Guenaga, Martin Corcoran, and Richard T. Wyatt. 2017. “Evolution of B Cell Analysis and Env Trimer Redesign.” Immunological Reviews 275 (1): 183-202.

Ke, Zunlong, Joaquin Oton, Kun Qu, Mirko Cortese, Vojtech Zila, Lesley McKeane, Takanori Nakane, et al. 2020. “Structures and Distributions of SARS-CoV-2 Spike Proteins on Intact Virions.” Nature 588 (7838): 498-502.

Koenig, Paul-Albert, Hrishikesh Das, Hejun Liu, Beate M. Kiimmerer, Florian N. Gohr, Lea-Marie Jenster, Lisa D. J. Schiffelers, et al. 2021. “Structure-Guided Multivalent Nanobodies Block SARS-CoV-2 Infection and Suppress Mutational Escape.” Science 371 (6530). https://doi.org/10.1126/science. ab e6230.

Korber, Bette, Will M. Fischer, Sandrasegaram Gnanakaran, Hyejin Yoon, James Theiler, Werner Abfalterer, Nick Hengartner, et al. 2020. “Tracking Changes in SARS-CoV-2 Spike: Evidence That D614G Increases Infectivity of the COVID-19 Virus.” Cell 182, 812-827.

Krammer, Florian. 2020. “SARS-CoV-2 Vaccines in Development.” Nature 586 (7830): 516-27.

Lainšček, Duško, Tina Fink, Vida Forstnerč, Iva Hafner-Bratkovič, Sara Orehek, Žiga Strmšek, Mateja Manček-Keber, et al. 2020. “Immune Response to Vaccine Candidates Based on Different Types of Nanoscaffolded RBD Domain of the SARS-CoV-2 Spike Protein.” Cold Spring Harbor Laboratory. https://doi.org/10.1101/2020.08.28.244269.

Liu, Lihong, Pengfei Wang, Manoj S. Nair, Jian Yu, Micah Rapp, Qian Wang, Yang Luo, et al. 2020. “Potent Neutralizing Antibodies against Multiple Epitopes on SARS-CoV-2 Spike.” Nature 584 (7821): 450-56.

Lu, Maolin, Pradeep D. Uchil, Wenwei Li, Desheng Zheng, Daniel S. Terry, Jason Gorman, Wei Shi, et al. 2020. “Real-Time Conformational Dynamics of SARS-CoV-2 Spikes on Virus Particles.” Cell Host & Microbe 28 (6): 880-91.e8.

McLellan, Jason S., Man Chen, M. Gordon Joyce, Mallika Sastry, Guillaume B. E. Stewart-Jones, Yongping Yang, Baoshan Zhang, et al. 2013. “Structure-Based Design of a Fusion Glycoprotein Vaccine for Respiratory Syncytial Virus.” Science 342 (6158): 592-98.

Pallesen, Jesper, Nianshuang Wang, Kizzmekia S. Corbett, Daniel Wrapp, Robert N. Kirchdoerfer, Hannah L. Turner, Christopher A. Cottrell, et al. 2017. “Immunogenicity and Structures of a Rationally Designed Prefusion MERS-CoV Spike Antigen.” Proceedings of the National Academy of Sciences of the United States of America 114 (35): E7348-57.

Planas, Delphine, Timothée Bruel, Ludivine Grzelak, Florence Guivel-Benhassine, Isabelle Staropoli, Francoise Porrot, Cyril Planchais, et al. 2021. “Sensitivity of Infectious SARS-CoV-2 B.1.1.7 and B.1.351 Variants to Neutralizing Antibodies.” Nature Medicine,

Polack, Fernando P., Stephen J. Thomas, Nicholas Kitchin, Judith Absalon, Alejandra Gurtman, Stephen Lockhart, John L. Perez, et al. 2020. “Safety and Efficacy of the BNT162b2 mRNA Covid-19 Vaccine.” The New England Journal of Medicine 383 (27): 2603-15.

Punjani, Ali, John L. Rubinstein, David J. Fleet, and Marcus A. Brubaker. 2017. “cryoSPARC: Algorithms for Rapid Unsupervised Cryo-EM Structure Determination.” Nature Methods 14 (3): 290-96.

Raska, Milan, Kazuo Takahashi, Lydie Czernekova, Katerina Zachova, Stacy Hall, Zina Moldoveanu, Matt C. Elliott, et al. 2010. “Glycosylation Patterns of HIV-1 gp120 Depend on the Type of Expressing Cells and Affect Antibody Recognition.” The Journal of Biological Chemistry 285 (27): 20860-69.

Robbiani, Davide F., Christian Gaebler, Frauke Muecksch, Julio C. C. Lorenzi, Zijun Wang, Alice Cho, Marianna Agudelo, et al. 2020. “Convergent Antibody Responses to SARS-CoV-2 in Convalescent Individuals.” Nature 584 (7821): 437-42.

Rogers, Thomas F., Fangzhu Zhao, Deli Huang, Nathan Beutler, Alison Burns, Wan-Ting He, Oliver Limbo, et al. 2020. “Isolation of Potent SARS-CoV-2 Neutralizing Antibodies and Protection from Disease in a Small Animal Model.” Science 369 (6506): 956-63.

Sacco, Michael Dominic, Chunlong Ma, Panagiotis Lagarias, Ang Gao, Julia Alma Townsend, Xiangzhi Meng, Peter Dube, et al. 2020. “Structure and Inhibition of the SARS-CoV-2 Main Protease Reveal Strategy for Developing Dual Inhibitors against Mpro and Cathepsin L.” Science Advances 6 (50). https://doi.org/10.1126/sciadv.abe0751.

Schoof, Michael, Bryan Faust, Reuben A. Saunders, Smriti Sangwan, Veronica Rezelj, Nick Hoppe, Morgane Boone, et al. 2020. “An Ultrapotent Synthetic Nanobody Neutralizes SARS-CoV-2 by Stabilizing Inactive Spike.” Science 370 (6523): 1473-79.

Shang, Jian, Yushun Wan, Chang Liu, Boyd Yount, Kendra Gully, Yang Yang, Ashley Auerbach, Guiqing Peng, Ralph Buie, and Fang Li. 2020. “Structure of Mouse Coronavirus Spike Protein Complexed with Receptor Reveals Mechanism for Viral Entry.” PLoS Pathogens 16 (3): e1008392.

Shang, Jian, Yushun Wan, Chuming Luo, Gang Ye, Qibin Geng, Ashley Auerbach, and Fang Li. 2020. “Cell Entry Mechanisms of SARS-CoV-2.” Proceedings of the National Academy of Sciences of the United States of America 117 (21): 11727-34.

Sliepen, Kwinten, Thijs van Montfort, Mark Melchers, Gözde Isik, and Rogier W. Sanders. 2015. “Immunosilencing a Highly Immunogenic Protein Trimerization Domain.” The Journal of Biological Chemistry 290 (12): 7436-42.

Stewart-Jones, Guillaume B. E., Paul V. Thomas, Man Chen, Aliaksandr Druz, M. Gordon Joyce, Wing-Pui Kong, Mallika Sastry, et al. 2015. “A Cysteine Zipper Stabilizes a Pre-Fusion F Glycoprotein Vaccine for Respiratory Syncytial Virus.” PloS One 10 (6): e0128779.

Sun, Xiangjie, Akila Jayaraman, Pavithra Maniprasad, Rahul Raman, Katherine V. Houser, Claudia Pappas, Hui Zeng, Ram Sasisekharan, Jacqueline M. Katz, and Terrence M. Tumpey. 2013. “N-Linked Glycosylation of the Hemagglutinin Protein Influences Virulence and Antigenicity of the 1918 Pandemic and Seasonal H1N1 Influenza A Viruses.” Journal of Virology 87 (15): 8756-66.

Sztain, Terra, Surl-Hee Ahn, Anthony T. Bogetti, Lorenzo Casalino, Jory A. Goldsmith, Ryan S. McCool, Fiona L. Kearns, et al. 2021. “A Glycan Gate Controls Opening of the SARS-CoV-2 Spike Protein.” Cold Spring Harbor Laboratory. https://doi.org/10.1101/2021.02.15.431212.

Tegunov, Dimitry, and Patrick Cramer. 2019. “Real-Time Cryo-Electron Microscopy Data Preprocessing with Warp.” Nature Methods 16 (11): 1146-52.

Tortorici, M. Alejandra, Martina Beltramello, Florian A. Lempp, Dora Pinto, Ha V. Dang, Laura E. Rosen, Matthew McCallum, et al. 2020. “Ultrapotent Human Antibodies Protect against SARS-CoV-2 Challenge via Multiple Mechanisms.” Science 370 (6519): 950-57.

Walls, Alexandra C., Young-Jun Park, M. Alejandra Tortorici, Abigail Wall, Andrew T. McGuire, and David Veesler. 2020. “Structure, Function, and Antigenicity of the SARS-CoV-2 Spike Glycoprotein.” Cell 181 (2): 281-92.e6.

Walls, Alexandra C., Xiaoli Xiong, Young-Jun Park, M. Alejandra Tortorici, Joost Snijder, Joel Quispe, Elisabetta Cameroni, et al. 2020. “Unexpected Receptor Functional Mimicry Elucidates Activation of Coronavirus Fusion.” Cell 183 (6): 1732.

Wang, Pengfei, Manoj S. Nair, Lihong Liu, Sho Iketani, Yang Luo, Yicheng Guo, Maple Wang, et al. 2021. “Antibody Resistance of SARS-CoV-2 Variants B.1.351 and B.1.1.7.” Nature, March. https://doi.org/10.1038/s41586-021-03398-2.

Watanabe, Yasunori, Joel D. Allen, Daniel Wrapp, Jason S. McLellan, and Max Crispin. 2020. “Site-Specific Glycan Analysis of the SARS-CoV-2 Spike.” Science 369 (6501): 330-33.

Wrapp, Daniel, Nianshuang Wang, Kizzmekia S. Corbett, Jory A. Goldsmith, Ching-Lin Hsieh, Olubukola Abiona, Barney S. Graham, and Jason S. McLellan. 2020. “Cryo-EM Structure of the 2019-nCoV Spike in the Prefusion Conformation.” Science 367 (6483): 1260-63.

Xiong, Xiaoli, Kun Qu, Katarzyna A. Ciazynska, Myra Hosmillo, Andrew P. Carter, Soraya Ebrahimi, Zunlong Ke, et al. 2020. “A Thermostable, Closed SARS-CoV-2 Spike Protein Trimer.” Nature Structural & Molecular Biology 27 (10): 934-41.

Xu, Cong, Yanxing Wang, Caixuan Liu, Chao Zhang, Wenyu Han, Xiaoyu Hong, Yifan Wang, et al. 2021. “Conformational Dynamics of SARS-CoV-2 Trimeric Spike Glycoprotein in Complex with Receptor ACE2 Revealed by Cryo-EM.” Science Advances 7 (1).

Yao, Hangping, Yutong Song, Yong Chen, Nanping Wu, Jialu Xu, Chujie Sun, Jiaxing Zhang, et al. 2020. “Molecular Architecture of the SARS-CoV-2 Virus.” Cell 183 (3): 730-38.e13.

Zhou, Daming, Wanwisa Dejnirattisai, Piyada Supasa, Chang Liu, Alexander J. Mentzer, Helen M. Ginn, Yuguang Zhao, et al. 2021. “Evidence of Escape of SARS-CoV-2 Variant B.1.351 from Natural and Vaccine-Induced Sera.” Cell,

Zhou, Tongqing, Yaroslav Tsybovsky, Jason Gorman, Micah Rapp, Gabriele Cerutti, Gwo-Yu Chuang, Phinikoula S. Katsamba, et al. 2020. “Cryo-EM Structures of SARS-CoV-2 Spike without and with ACE2 Reveal a pH-Dependent Switch to Mediate Endosomal Positioning of Receptor-Binding Domains.” Cell Host & Microbe 28 (6): 867-79.e5.

### REFERENCES—EXAMPLE 2

Mamo T, Poland GA. Nanovaccinology: The next generation of vaccines meets 21st century materials science and engineering. Vaccine. 2012;30(47):6609-6611. [PubMed] [Google Scholar]

des Rieux A, Fievez V, Garinot M, Schneider Y J, Pr√\textcircled{c}at V. Nanoparticles as potential oral delivery systems of proteins and vaccines: A mechanistic approach. Journal of Controlled Release. 2006; 116(1): 1-27. [PubMed] [Google Scholar]

Singh M, Chakrapani A, O'Hagan D. Nanoparticles and micro-particles as vaccine-delivery systems. Expert Review of Vaccines. 2007;6(5):797-808. [PubMed] [Google Scholar]

Oyewumi M O, Kumar A, Cui Z. Nano-microparticles as immune adjuvants: Correlating particle sizes and the resultant immune responses. Expert Review of Vaccines. 2010;9(9):1095-1107. [PMC free article][PubMed] [Google Scholar]

Zhao L, Seth A, Wibowo N, Zhao C X, Mitter N, Yu C, Middelberg AP. Nanoparticle vaccines. Vaccine. 2014;32(3):327-337. [PubMed] [Google Scholar]

Zhao K, Chen G, Shi X, Gao T, Li W, Zhao Y, Zhang F, Wu J, Cui X, Wang Y F. Preparation and efficacy of a live newcastle disease virus vaccine encapsulated in chitosan nanoparticles. PLoS One. 2012;7(12):e53314. [PMC free article] [PubMed] [Google Scholar]

Borges O, Cordeiro-da-Silva A, Tavares J, Santar√\textcircled{c}m N, de Sousa A, Borchard G, Junginger HE. Immune response by nasal delivery of hepatitis B surface antigen and codelivery of a CpG ODN in alginate coated chitosan nanoparticles. European Journal of Pharmaceutics and Biopharmaceutics. 2008;69(2):405-416. [PubMed] [Google Scholar]

Nochi T, Yuki Y, Takahashi H, Sawada S, Mejima M, Kohda T, Harada N, Kong I G, Sato A, Kataoka N, et al. Nanogel antigenic protein-delivery system for adjuvant-free intranasal vaccines. Nature Materials. 2010;9(7):572-578. [PubMed] [Google Scholar]

Stone J W, Thornburg N J, Blum D L, Kuhn S J, Wright D W, Crowe J E., Jr Gold nanorod vaccine for respiratory syncytial virus. Nanotechnology. 2013;24(29):295102. [PMC free article] [PubMed] [Google Scholar]

Wang T, Zou M, Jiang H, Ji Z, Gao P, Cheng G. Synthesis of a novel kind of carbon nanoparticle with large mesopores and macropores and its application as an oral vaccine adjuvant. European Journal of Pharmaceutical Sciences. 2011;44(5):653-659. [PubMed] [Google Scholar]

Gl√°ck R, Moser C, Metcalfe I C. Influenza virosomes as an efficient system for adjuvanted vaccine delivery. Expert Opinion on Biological Therapy. 2004;4(7):1139-1145. [PubMed] [Google Scholar]

Zhu F C, Zhang J, Zhang X F, Zhou C, Wang Z Z, Huang S J, Wang H, Yang C L, Jiang H M, Cai J P, et al. Efficacy and safety of a recombinant hepatitise vaccine in healthy adults: A large-scale, randomised, double-blind placebo-controlled, phase 3 trial. Lancet. 2010;376(9744):895-902. [PubMed] [Google Scholar]

Sliepen K, Ozorowski G, Burger J A, van Montfort T, Stunnenberg M, LaBranche C, Montefiori D C, Moore J P, Ward A B, Sanders R W. Presenting native-like HIV-1 envelope trimers on ferritin nanoparticles improves their immunogenicity. Retrovirology. 2015;12(82):15-21. [PMC free article] [PubMed] [Google Scholar]

Champion C I, Kickhoefer V A, Liu G, Moniz R J, Freed A S, Bergmann L L, Vaccari D, Raval-Fernandes S, Chan A M, Rome L H, Kelly K A. A vault nanoparticle vaccine induces protective mucosal immunity. PLoS One. 2009;4(4):e5409. [PMC free article] [PubMed] [Google Scholar]

Kanekiyo M, Wei C J, Yassine H M, McTamney P M, Boyington J C, Whittle J R, Rao S S, Kong W P, Wang L, Nabel G J. Self-assembling influenza nanoparticle vaccines elicit broadly neutralizing H1N1 antibodies. Nature. 2013;499(7456):102-106. [PMC free article] [PubMed] [Google Scholar]

Cho K J, Shin H J, Lee J H, Kim K J, Park S S, Lee Y, Lee C, Park S S, Kim K H. The crystal structure of ferritin from helicobacter pylori reveals unusual conformational changes for iron uptake. Journal of Molecular Biology. 2009;390(1):83-98. [PubMed] [Google Scholar]

Steinman RM. Decisions about dendritic cells: Past, present, and future. Annual Review of Immunology. 2012;30(1):1-22. [PubMed] [Google Scholar]

Gilboa E. DC-based cancer vaccines. Journal of Clinical Investigation. 2007;117(5):1195-1203. [PMC free article] [PubMed] [Google Scholar]

Aarntzen E, Figdor C, Adema G, Punt C, De Vries I. Dendritic cell vaccination and immune monitoring. Cancer Immunology, Immunotherapy. 2008;57(10):1559-1568. [PMC free article] [PubMed] [Google Scholar]

Han J A, Kang Y J, Shin C, Ra J S, Shin H H, Hong S Y, Do Y, Kang S. Ferritin protein cage nanoparticles as versatile antigen delivery nanoplatforms for dendritic cell (DC)-based vaccine development. Nanomedicine; Nanotechnology, Biology, and Medicine. 2013;10(3):561-569. [PubMed] [Google Scholar]

Shimonkevitz R, Colon S, Kappler J W, Marrack P, Grey H M. Antigen recognition by H-2-restricted T cells. II. A tryptic ovalbumin peptide that substitutes for processed antigen. Journal of Immunology (Baltimore, MD: 1950) 1984;133(4):2067-2074. [PubMed] [Google Scholar]

What is claimed is:

1. A mutant coronavirus spike protein comprising at least one of the following modifications:
(1) a short flexible peptide linker or a rigid peptide linker in place of a furin cleavage site loop to genetically link an Si and S2 subunit;
(2) at least one additional disulfide bond; and
(3) 1, 2, 3, 4, or 5 proline mutations for greater trimeric stability, wherein the mutant coronavirus spike protein has at least one of: a higher stability or a higher level of expression when compared to a non-modified coronavirus spike protein.

2. The mutant coronavirus spike protein of claim 1, wherein the furin cleavage site loop is at position 676-690.

3. The mutant coronavirus spike protein of claim 1, wherein the short flexible peptide linker is selected from at least one of: GGS (SEQ ID NO:34), GP (SEQ ID NO:35), GPGP (SEQ ID NO:36), GGSGGS (SEQ ID NO:37), or GGGSGGGS (SEQ ID NO:38).

4. The mutant coronavirus spike protein of claim 1, wherein the 1, 2, 3, 4, or 5 proline mutations are selected from F817P, A892P, A899P, A942P, P986K, K986P, V987P, and P987V.

5. The mutant coronavirus spike protein of claim 1, wherein the at least one additional disulfide bond is selected from F43C-G566C, G413C-P987C, Y707C-T883C, G1035C-V1040C, A701C-Q787C, G667C-L864C, V382C-R983C, and 1712C-I816C.

6. The mutant coronavirus spike protein of claim 1, wherein the proline mutations are not K986P and V987P mutations.

7. The mutant coronavirus spike protein of claim 1, wherein the at least one addition disulfide bond links the S2 to S2′ subunit, the S1 to S2 subunit, or the S1 to S2′ subunit.

8. The mutant coronavirus spike protein of claim 1, wherein the higher stability is selected from: increased temperature stability, increased freeze/thaw stability, or increased lyophilization/resuspension stability.

9. The mutant coronavirus spike protein of claim 1, further comprising a purification peptide at an amino-terminus, a carboxy-terminus, or both.

10. The mutant coronavirus spike protein of claim 1, wherein the mutant coronavirus spike protein is selected from SEQ ID NOS:1 to 32.

11. The mutant coronavirus spike protein of any one of claims 1-10, wherein the coronavirus is SARS, MERS, 229E (alpha), NL63 (alpha), OC43 (beta), HKU1 (beta), SARS-CoV-2, or an emerging variant thereof.

12. The mutant coronavirus spike protein of any one of claims 1-11, wherein the coronavirus is SARS-CoV-2.

13. A nucleic acid encoding the mutant coronavirus spike protein of any one of claims 1-12.

14. The nucleic acid of claim 13, further comprising a vector.

15. A cell comprising the mutant coronavirus spike protein of any one of claims 1-12 or the nucleic acid of claim 13.

16. The cell of claim 15, wherein the cell is a human cell.

17. A vaccine composition comprising the mutant coronavirus spike protein of any one of claims 1-12 or nucleic acid of either claim 13 or claim 14, and a pharmaceutically acceptable excipient.

18. The vaccine composition of claim 17, further comprising an adjuvant.

19. A nanoparticle comprising the mutant coronavirus spike protein of any one of claims 1-12.

20. The nanoparticle of claim 19, wherein the nanoparticle comprises at least two of the mutant coronavirus spike protein of any one of claims 1-12.

21. The nanoparticle of claim 19, wherein the mutant coronavirus spike proteins are formed into dimers, trimers, or multimers.

22. The nanoparticle of any one of claims 19-21, wherein the nanoparticles comprise ferritin nanoparticles, polymeric nanoparticles, or both.

23. A method of making a mutant coronavirus spike protein comprising:
obtaining a nucleic acid sequence encoding a coronavirus spike protein; and
modifying the nucleic acid sequence of the coronavirus spike protein such that the amino acid sequence expressed by the nucleic acid sequence comprises at least one of:
linking the S1/S2 subunits of a coronavirus spike protein, by deleting a furin cleavage site loop and adding a short flexible peptide linker or a rigid peptide linker;
adding at least one additional disulfide bond; and
adding 1, 2, 3, 4, or 5 proline mutations for greater trimeric stability, wherein the expressed mutant coronavirus spike protein has at least one of: a higher stability or level of expression, than a non-modified coronavirus spike protein.

24. The method of claim 23, further comprising the step of expressing the mutant coronavirus spike protein in a bacteria, fungi, mammalian cell, avian cell, insect cell, or plant cell.

25. The method of claim 23, wherein the furin cleavage site loop is at position 676-690.

26. The method of claim 23, wherein the linker is selected from at least one of: GGS (SEQ ID NO:34), GP (SEQ ID NO:35), GPGP (SEQ ID NO:36), GGSGGS (SEQ ID NO:37), or GGGSGGGS (SEQ ID NO:38).

27. The method of claim 23, wherein the 1, 2, 3, 4, or 5 proline mutations are selected from F817P, A892P, A899P, A942P, P986K, K986P, V987P, and P987V.

28. The method of claim 23, wherein the at least one additional disulfide bond is selected from F43C-G566C, G413C-P987C, Y707C-T883C, G1035C-V1040C, A701C-Q787C, G667C-L864C, V382C-R983C, or I712C-I816C.

29. The method of claim 23, wherein the proline mutations are not K986P and V987P mutations.

30. The method of claim 23, wherein the at least one addition disulfide bond links the S2 to S2′ subunit, the S1 to S2 subunit, or the S1 to S2′ subunit.

31. The method of claim 23, wherein the higher stability is selected from: increased temperature stability, increased freeze/thaw stability, or increased lyophilization/resuspension stability.

32. The method of claim 23, further comprising including a purification peptide at an amino-terminus, a carboxy-terminus, or both.

33. The method of claim 23, wherein the mutant coronavirus spike protein expressed by the modified nucleic acid sequence is selected from SEQ ID NOS:1 to 32.

34. The method of any one of claims 23, wherein the coronavirus is SARS, MERS, 229E (alpha), NL63 (alpha), OC43 (beta), HKU1 (beta), SARS-CoV-2, or an emerging variant thereof.

35. A method of immunizing a subject in need thereof against a coronavirus, the method comprising:
identifying a subject in need of an immunization; and
administering to the subject to a mutant coronavirus spike protein comprising at least one of the following modifications:
a short flexible peptide linker or a rigid peptide linker in place of a furin cleavage site loop to genetically link an S1 and S2 subunit;
at least one additional disulfide bond; and
1, 2, 3, 4, or 5 proline mutations for greater trimeric stability, wherein the mutant coronavirus spike protein has at least one of: a higher stability or a higher level of expression when compared to a non-modified coronavirus spike protein.

36. The method of claim 35, wherein administering the mutant coronavirus spike protein comprises administering the vaccine composition of either of claim 17 or claim 18.

37. The method of claim 35, wherein the mutant coronavirus spike protein has an amino acid sequence corresponding to any one of SEQ ID NOS:1 to 33.

38. The method of claim 35, wherein the coronavirus is SARS, MERS, 229E (alpha), NL63 (alpha), OC43 (beta), HKU1 (beta), SARS-CoV-2, or an emerging variant thereof.

39. The method of claim 35, further comprising isolating B cells from the immunized subject and obtaining a nucleic acid sequence of antibodies from the B cells, or fusing the isolated B cells with an immortalized cell to make a hybridoma.

40. A nucleic acid sequence encoding a mutant coronavirus spike protein comprising:
one or more mutations that change an amino acid sequence of a coronavirus spike protein by at least one of:
linking the S1/S2 subunits of a coronavirus spike protein, by deleting a furin cleavage site loop and adding a short flexible peptide linker or a rigid peptide linker;
adding at least one additional disulfide bond; or
adding 1, 2, 3, 4, or 5 proline mutations for greater trimeric stability, wherein the mutant coronavirus spike protein has at least one of: higher stability or level of expression, than a non-modified coronavirus spike protein.

41. The nucleic acid of claim 40, wherein the coronavirus is SARS, MERS, 229E (alpha), NL63 (alpha), OC43 (beta), HKU1 (beta), SARS-CoV-2, or an emerging variant thereof.

42. A vector comprising a nucleic acid sequence encoding a mutant coronavirus spike protein comprising:
one or more mutations that change the encoded amino acid sequence by at least one of:
linking the S1/S2 subunits of a coronavirus spike protein, by deleting a furin cleavage site loop and adding a short flexible peptide linker or a rigid peptide linker;
adding at least one additional disulfide bond; and
adding 1, 2, 3, 4, or 5 proline mutations for greater trimeric stability, wherein the mutant coronavirus spike protein has at least one of: higher stability or level of expression, than a non-modified coronavirus spike protein.

43. The vector of claim 42, where the vector is selected for expression in a bacteria, fungi, mammalian cell, avian cell, insect cell, or plant cell.

44. The vector of claim 42, where the vector is in a bacteria, fungi, mammalian cell, avian cell, insect cell, or plant cell.

45. The vector of claim 42, wherein the coronavirus is SARS, MERS, 229E (alpha), NL63 (alpha), OC43 (beta), HKU1 (beta), SARS-CoV-2, or an emerging variant thereof.
